(19) United States
(12) Patent Application Publication
(10) Pub. No.: US 2021/0000792 A1
(43) Pub. Date: Jan. 7, 2021

(54) METHODS FOR REDUCING OR PREVENTING CARDIOVASCULAR EVENTS IN PATIENTS WITH TYPE II DIABETES MELLITUS

(71) Applicant: Janssen Pharmaceutica NV, Beerse (BE)

(72) Inventors: Norman R. Rosenthal, Hopewell, NJ (US); Douglas K. Ways, Ringoes, NJ (US)

(21) Appl. No.: 17/021,370

(22) Filed: Sep. 15, 2020

Related U.S. Application Data
(63) Continuation of application No. 16/004,607, filed on Jun. 11, 2018.
(60) Provisional application No. 62/518,547, filed on Jun. 12, 2017.

(51) Int. Cl. A61K 31/381; A61P 9/00; A61P 3/00; A61K 31/7042

(52) U.S. Cl. CPC A61K 31/381; A61K 31/7042; A61P 3/00; A61P 9/00

(57) ABSTRACT

The present invention is directed to methods for reducing, preventing or slowing the progression of cardiovascular risk factors and/or cardiovascular disease, comprising administration of canagliflozin.

### CROSS-REFERENCE TO RELATED APPLICATIONS

This application claims priority to U.S. Provisional Patent Application No. 62/518,547, filed Jun. 12, 2017, the disclosure of which is hereby incorporated by reference in its entirety.

### FIELD OF THE INVENTION

The present invention is directed to methods for reducing, preventing or slowing the progression of cardiovascular risk factors and/or cardiovascular disease, comprising administration of canagliflozin.

### BACKGROUND OF THE INVENTION

Cardiovascular disease (CVD) is a class of diseases that involve the heart or blood vessel.

There are many cardiovascular diseases involving the blood vessels. They are known as vascular diseases and include: coronary artery disease (also known as coronary heart disease and ischemic heart disease, and including, but not limited to angina, myocardial infarction, etc.), peripheral arterial disease (disease of blood vessels that supply blood to the arms and legs), cerebrovascular disease (disease of blood vessels that supply blood to the brain, including stroke or ischemia), renal artery stenosis, aortic aneurysm.

There are also many cardiovascular diseases that involve the heart include cardiomyopathy, (diseases of cardiac muscle), hypertensive heart disease (diseases of the heart secondary to high blood pressure or hypertension), heart failure (clinical syndrome caused by the inability of the heart to supply sufficient blood to the tissues to meet their metabolic requirements), pulmonary heart disease (a failure at the right side of the heart with respiratory system involvement), cardiac dysrhythmias (abnormalities of heart rhythm), inflammatory heart disease, endocarditis (inflammation of the inner layer of the heart, the endocardium, most commonly involving the heart valves), inflammatory cardiomegaly, myocarditis (inflammation of the myocardium, the muscular part of the heart), valvular heart disease, congenital heart disease (heart structure malformations existing at birth), rheumatic heart disease (heart muscles and valves damage due to rheumatic fever).

The underlying mechanisms vary depending on the disease and there are many risk factors for heart diseases: age, gender, tobacco use, physical inactivity, excessive alcohol consumption, unhealthy diet, obesity, genetic predisposition and family history of cardiovascular disease, raised blood pressure (hypertension), raised blood sugar (including Type diabetes mellitus), raised blood cholesterol (hyperlipidemia), psychosocial factors, poverty and low educational status, and air pollution. While the individual contribution of each risk factor varies between different communities or ethnic groups the overall contribution of these risk factors is very consistent. Some of these risk factors, such as age, gender or family history/genetic predisposition, are immutable; however, many important cardiovascular risk factors are modifiable by lifestyle change, social change, drug treatment (for example prevention of hypertension, hyperlipidemia, and diabetes).

Coronary artery disease, stroke, and peripheral artery disease involve atherosclerosis, which in turn may be caused by high blood pressure, smoking, diabetes, lack of exercise, obesity, high blood cholesterol, poor diet and excessive alcohol consumption, among others. High blood pressure results in 13% of CVD deaths, while tobacco results in 9%, diabetes 6%, lack of exercise 6% and obesity 5%.

Existing cardiovascular disease or a previous cardiovascular event, such as a heart attack or stroke, is the strongest predictor of a future cardiovascular event. Age, sex, smoking, blood pressure, blood lipids and diabetes are important predictors of future cardiovascular disease in people who are not known to have cardiovascular disease. These measures, and sometimes others, may be combined into composite risk scores to estimate an individual's future risk of cardiovascular disease. Numerous risk scores exist although their respective merits are debated. Other diagnostic tests and biomarkers remain under evaluation but currently these lack clear-cut evidence to support their routine use. They include family history, coronary artery calcification score, high-sensitivity C-reactive protein (hs-CRP), ankle-brachial pressure index, lipoprotein subclasses and particle concentration, lipoprotein(a), apolipoproteins A-I and B, fibrinogen, white blood cell count, homocysteine, N-terminal pro B-type natriuretic peptide (NT-proBNP), and markers of kidney function. High blood phosphorous is also linked to an increased risk.

It is estimated that 90% of CVD is preventable. Prevention of atherosclerosis involves improving risk factors through: healthy eating, exercise, avoidance of tobacco smoke and limiting alcohol intake. Treating risk factors, such as high blood pressure, blood lipids and diabetes is also beneficial. The effect of the use of aspirin in people who are otherwise healthy is of unclear benefit.

Cardiovascular diseases are the leading cause of death globally. This is true in all areas of the world except Africa. Together they resulted in 17.9 million deaths (32.1%) in 2015 up from 12.3 million (25.8%) in 1990. Coronary artery disease and stroke account for 80% of CVD deaths in males and 75% of CVD deaths in females. Most cardiovascular disease affects older adults. In the United States 11% of people between 20 and 40 have CVD, while 37% between 40 and 60, 71% of people between 60 and 80, and 85% of people over 80 have CVD.

There remains a need for additional safe and effective treatments for patients with Type II diabetes mellitus and concomitant or comorbid cardiovascular disease or risk of cardiovascular disease.

### SUMMARY OF THE INVENTION

The present invention is directed to methods for reducing or preventing one or more cardiovascular events comprising administering to a patient in need thereof a therapeutically effective amount of canagliflozin; wherein the patient in need thereof is a patient diagnosed with Type II diabetes mellitus; and wherein the patient further exhibits symptoms of or is diagnosed with one or more concomitant or comorbid cardiovascular risk factors or cardiovascular disease.

The present invention is directed to methods for reducing or preventing one or more MACE (major adverse cardiac event) comprising administering to a patient in need thereof a therapeutically effective amount of canagliflozin; wherein the patient in need thereof is a patient diagnosed with Type II diabetes mellitus; and wherein the patient further exhibits symptoms of or is diagnosed with one or more concomitant or comorbid cardiovascular risk factors or cardiovascular disease.

The present invention is further directed to a method of treating a patient at increased risk of major adverse cardiovascular event (MACE) comprising: selecting for treatment a patient at increased risk of MACE; and administering to said patient a therapeutically effective amount of canagliflozin; wherein the patient at increased risk of MACE has further been diagnosed with Type II diabetes mellitus; and wherein the therapeutically effective amount of canagliflozin is sufficient to reduce said increased risk of MACE.

The present invention is further directed to methods for reducing or preventing one or more cardiovascular events comprising administering to a patient in need thereof a therapeutically effective amount of canagliflozin;

wherein the patient in need thereof is a patient diagnosed with Type II diabetes mellitus; and wherein the patient further exhibits symptoms of or is diagnosed with one or more concomitant or comorbid cardiovascular risk factors or cardiovascular disease.

and wherein the cardiovascular event (to be reduced or prevented) is selected from the group consisting of cardiovascular hospitalization, non-fatal myocardial infarction, non-fatal ischemia or stroke, and cardiovascular mortality (including but not limited to sudden cardiac death).

The present invention is further directed to methods for reducing or preventing one or more cardiovascular events comprising administering to a patient in need thereof a therapeutically effective amount of canagliflozin;

wherein the patient in need thereof is a patient diagnosed with Type II diabetes mellitus; wherein the patient is further diagnosed with microalbuminuria (ACR≥30 mg/g and ≤300 mg/g) or macroalbuminuria (ACR>300 mg/g); and wherein the patient further exhibits symptoms of or is diagnosed with one or more concomitant or comorbid cardiovascular risk factors or cardiovascular disease;

and wherein the cardiovascular event (to be reduced or prevented) is selected from the group consisting of cardiovascular hospitalization, non-fatal myocardial infarction, non-fatal ischemia or stroke, and cardiovascular mortality (including but not limited to sudden cardiac death).

### BRIEF DESCRIPTION OF THE FIGURES

FIG. 1 illustrates a flowchart detailing the prespecified hypothesis testing plan for evaluating cardiovascular outcomes.

FIG. 2a illustrates effect of canagliflozin on glycated hemoglobin.

FIG. 2b illustrates the effect of canagliflozin on body weight.

FIG. 2c illustrates the effect of canagliflozin on systolic blood pressure.

FIG. 2d illustrates the effect of canagliflozin on diastolic blood pressure.

FIG. 3 illustrates the effects of canagliflozin on cardiovascular, renal, hospitalization and death outcomes.

FIGS. 4a) through 4h) illustrate the effect of canagliflozin on cardiovascular and renal outcomes, more particularly,

FIG. 4a illustrates the effect of canagliflozin on cardiovascular death, non-fatal stroke or non-fatal myocardial infarction.

FIG. 4b illustrates the effect of canagliflozin on cardiovascular death.

FIG. 4c illustrates the effect of canagliflozin on non-fatal stroke.

FIG. 4d illustrates the effect of canagliflozin on non-fatal myocardial infarction.

FIG. 4e illustrates the effect of canagliflozin on hospitalized heart failure.

FIG. 4f illustrates the effect of canagliflozin on all-cause mortality.

FIG. 4g illustrates the effect of canagliflozin on progression of albuminuria.

FIG. 4h illustrates the effect of canagliflozin on renal composite.

### DETAILED DESCRIPTION OF THE INVENTION

The present invention is directed to methods for reducing or preventing one or more cardiovascular events comprising administering to a patient in need thereof a therapeutically effective amount of canagliflozin, as described in more detail herein.

In certain embodiments, the present invention is directed to methods for reducing or preventing a cardiovascular event, cardiovascular hospitalization, non-fatal myocardial infarction, non-fatal ischemic events or strokes, or cardiovascular mortality. In certain embodiments, the present invention is directed to methods for reducing or preventing one or more MACE (major adverse cardiac event).

In certain embodiments, the present invention is directed to methods for reducing or preventing hospitalizations due to cardiovascular symptoms or events. In certain embodiments, the present invention is directed to methods for preventing at least about 2%, 3%, 5%, 10%, 12%, 15%, 18%, 20%, 22%, 25%, 28% or 30% of cardiovascular hospitalizations in patients diagnosed with TYPE II Diabetes Mellitus and one or more concomitant or comorbid cardiovascular risk factors or cardiovascular disease.

In certain embodiments, the present invention is directed to methods for reducing or preventing non-fatal myocardial infarction. In certain embodiments, the present invention is directed to methods for preventing at least about 2%, 3%, 5%, 10%, 12%, 15%, 18%, 20%, 22%, 25%, 28% or 30% of non-fatal myocardial infarctions in patients diagnosed with TYPE II Diabetes Mellitus and one or more concomitant or comorbid cardiovascular risk factors or cardiovascular disease.

In certain embodiments, the present invention is directed to methods for reducing or preventing non-fatal ischemic events or strokes. In certain embodiments, the present invention is directed to methods preventing at least about 2%, 3%, 5%, 10%, 12%, 15%, 18%, 20%, 22%, 25%, 28% or 30% of non-fatal ischemic events or strokes in patients diagnosed with TYPE II Diabetes Mellitus and one or more concomitant or comorbid cardiovascular risk factors or cardiovascular disease.

In certain embodiment, the present invention is directed to methods for reducing or preventing cardiovascular mortality. In certain embodiments, the present invention is directed to methods for preventing at least about 2%, 3%, 5%, 10%, 12%, 15%, 18%, 20%, 22%, 25%, 28% or 30% of cardiovascular mortality in patients diagnosed with TYPE II Diabetes Mellitus and one or more concomitant or comorbid cardiovascular risk factors or cardiovascular disease.

In certain embodiments, the present invention is directed to a safe and effective method for treating a patient with Type II diabetes mellitus comprising administering to said patient a therapeutically effective amount of canagliflozin.

In certain embodiments, the present invention is directed to a method of decreasing a patient's risk of cardiovascular hospitalizations, cardiovascular events or cardiovascular mortality, comprising administrating to said patient a therapeutically effective amount of canagliflozin;

wherein said patient is diagnosed with Type II diabetes mellitus; and wherein said patient is further diagnosed with or exhibits symptoms of one or more cardiovascular risk factors selected from the group consisting of hypertension or high blood pressure (for example, elevated systolic blood pressure, elevated diastolic blood pressure, or a blood pressure of greater than 140/90 mm Hg, preferably greater than about 145/95 mm Hg), elevated cholesterol (hyperlipidemia), elevated LDL, depressed HDL levels, elevated triglycerides, obesity (as defined by for example, a BMI of greater than 30, preferably, morbid obesity defined by a BMI of greater than 40), cardiovascular disease (for example, previous myocardial infarction, angina, heart failure, stroke), microalbuminuria (as defined for example by ACR≥30 mg/g and ≤300 mg/g), macroalbuminuria (as defined by for example ACR>300 mg/g), peripheral vascular disease (for example, carotid stenosis, femoral artery stenosis, current or past smoking, family history of cardiovascular disease and male gender.

In certain embodiments, the present invention is directed to methods for preventing or reducing cardiovascular events in a patient with heart failure (including Class I through Class IV, preferably Class II through Class IV, more preferably Class III or Class IV), wherein heart failure is indicated by one or more of the following:
a) a history of, or current symptoms of congestive heart failure;
b) symptoms of heart failure with minimal exertion;
c) hospitalization of the patient for heart failure;
d) hospitalization of the patient for NYHA Class IV heart failure;
e) hospitalization of the patient for NYHA Class III heart failure;
f) hospitalization of the patient for NYHA Class II heart failure
g) hospitalization of the patient for NYHA Class I heart failure; or
h) hospitalization of the patient for heart failure with recent decompensation requiring hospitalization or intravenous therapy for the treatment of heart failure.

In certain embodiments, the present invention is directed to methods for preventing or reducing cardiovascular events in a patient with congestive heart failure, wherein the congestive heart failure is:
a) congestive heart failure in a stable hemodynamic condition;
b) congestive heart failure defined by a reduced left ventricular ejection fraction below 0.35 in a stable hemodynamic condition;
c) congestive heart failure defined as NYHA Class I in a stable hemodynamic condition;
d) congestive heart failure defined as NYHA Class II in a stable hemodynamic condition;
e) congestive heart failure defined as NYHA Class III in a stable hemodynamic condition; or
f) congestive heart failure defined as NYHA Class IV in a stable hemodynamic condition.

In certain embodiments, the present invention is directed to methods for preventing or reducing cardiovascular events in a patient diagnosed with Type II Diabetes Mellitus and concomitant or comorbid congestive heart failure (including, for example, NYHA class IV, NYHA class III, NYHA class II and NYHA class I).

In certain embodiments, the present invention is directed to methods for preventing or reducing cardiovascular events in a patient with heart failure in an unstable hemodynamic condition, wherein heart failure in an unstable hemodynamic condition may be defined by any of the following:
a) worsening symptoms of heart failure at rest or with minimal exertion;
b) history of, or current symptoms of congestive heart failure at rest;
c) symptoms of heart failure with minimal exertion within the last month, i.e. the month prior to start of treatment or, hospitalization for heart failure;
d) NYHA Class IV;
e) NYHA Class III;
f) NYHA Class II;
g) NYHA Class I; or
h) recent decompensation requiring hospitalization or intravenous therapy for the treatment of heart failure.

In certain embodiments, the present invention is directed to methods which favorably modulate (or improve) one or more diagnostic indicators predictive of a major adverse cardiovascular event. There are a large number of such diagnostic indicators, which include, for example, blood pressure, treadmill testing, troponin testing, fluid volume, cardiac output, ejection fraction, cardiomyopathy, cardiac hypertrophy, ECG abnormalities, external oxygen dependence, diuretic requirements, hospitalization for cardiac insufficiency, unstable plaque, angina, arrhythmias, Q-T interval, elevated triglycerides, elevated LDL, or low HDL, and the like. In certain embodiments, such a favorable modulation of a diagnostic indicator predictive of a major adverse cardiovascular event in a patient can be a modulation of at least or at least about 5%, 10%, 15%, 20%, 25%, 30%, 35%, 40%, 45%, 50%, 60%, 70%, 80%, or 90%, relative to the diagnostic indicator predictive of a major adverse cardiovascular event in a patient at the same level of risk of MACE, but who is not receiving treatment by administration of canagliflozin according to the methods provided herein.

In certain embodiments, the methods of the present invention result in a patient's hazard ratio (HR) (comparing risk in treated group versus risk in placebo group per industry standards) for a particular cardiovascular event or outcome, for example, cardiovascular death, nonfatal myocardial infarction, stroke, fatal stroke, nonfatal stroke, nonfatal HUSA (hospitalization due to unstable angina), coronary revascularization procedure, and/or all-cause mortality, in a range of from about 1.0 to about 0.50, or any amount or range therein, preferably in the range of from about 0.90 to about 0.60, more preferably in a range of from about 0.90 to about 0.75, more preferably in a range of from about 0.85 to about 0.65, for example, less than about 1.0, 0.99, 0.98, 0.97, 0.96, 0.95, 0.94, 0.93, 0.92, 0.91, 0.90, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51 or 0.50 or any range defined by any two of the preceding values.

In certain embodiments, one or more improvements provided by the methods of the present invention (e.g. reduction in the risk of one or more MACE, reduction in the predicted severity of an adverse cardiovascular event, decrease in the predicted mortality from an adverse cardio-vascular event, decrease in the progression of cardiovascular disease in a patient, increase in the predicted life expectancy of the patient, or increase in the predicted time period until next occurrence of an adverse cardiovascular event, the increase in the effectiveness of a cardiovascular intervention in a patient, or favorable modulation in a diagnostic indicator predictive of a major adverse cardiovascular event), may continue for a period of time after the discontinuation of the administration of canagliflozin. In certain embodiments, this period of time is, or is at least, about 1, 2, 3, 4, 5, or 6 months, or 0.5, 1, 2, 3, 4, or 5 years, or between 1-6 months, 1 month to 1 year, 4 months to 2 years, or 6 months to 5 years.

In certain embodiments of the present invention, one or more improvements provided by the methods of the present invention (e.g. reduction in the risk of one or more MACE, reduction in the predicted severity of an adverse cardiovascular event, decrease in the predicted mortality from an adverse cardiovascular event, decrease in the progression of cardiovascular disease in a patient, increase in the predicted life expectancy of the patient, or increase in the predicted time period until next occurrence of an adverse cardiovascular event, the increase in the effectiveness of a cardiovascular intervention in a patient, or favorable modulation in a diagnostic indicator predictive of a major adverse cardiovascular event), is seen in a treated patient population as compared to a control population, for example between patients receiving canagliflozin and patients receiving placebo. In certain embodiments, improvement can be observed between the two patient populations in, or in as few as about 24 weeks (in for example MACE outcomes), preferably in as few as about 6-12 weeks.

In certain embodiments of the present invention, the patient in need thereof is a patient diagnosed with Type II diabetes mellitus; and further exhibits symptoms of or is diagnosed with one or more concomitant or comorbid cardiovascular risk factors or cardiovascular disease.

In certain embodiments of the present invention, the patient diagnosed with Type II diabetes mellitus has a measured HbA1c in the range of ≥7.0% and ≤10.5%.

In certain embodiments of the present invention, the patient is over 30 years of age and has a history of at least non-fatal myocardial infarction, non-fatal stoke or has a history of symptomatic atherosclerotic vascular disease. In certain embodiment of the present invention, the patient is over 50 years of age and exhibits or presents with two or more risk factors of vascular disease (including but not limited to elevated urinary albumin:creatinine ratio).

In certain embodiments of the present invention, the one or more cardiovascular risk factors are independently selected from the group consisting of high blood pressure (for example, elevated systolic blood pressure, elevated diastolic blood pressure, or a blood pressure of greater than about 145/95 mm Hg), elevated cholesterol (hyperlipidemia), elevated LDL, depressed HDL levels, elevated triglycerides, obesity (as defined by for example, a BMI of greater than 30, preferably, morbid obesity defined by a BMI of greater than 40), cardiovascular disease (for example, previous myocardial infarction, angina, heart failure, stroke), microalbuminuria (as defined for example by ACR≥30 mg/g and ≤300 mg/g), macroalbuminuria (as defined by for example ACR>300 mg/g), peripheral vascular disease (for example, carotid stenosis, femoral artery stenosis, or current or past smoking, family history of cardiovascular disease or male gender.

In certain embodiments of the present invention, the patient has a measured eGFR greater than about 30 ms/min/1.73 m², preferably greater than about 60 mls/min/1.73 m². In certain embodiments of the present invention, the patient has a measured eGFR less than about 90 ms/min/1.73 m² and greater than about 60 ms/min/1.73 m².

In certain embodiments of the present invention, the cardiovascular disease is selected from the group consisting of heart failure (including but not limited to congestive heart failure), cardiac arrhythmia, atrial fibrillation, ventricular fibrillation, tachyarrhythmia (not sinus tachycardia), angina (including but not limited to unstable angina) and hypertension.

In certain embodiments of the present invention, the patient has a history of one or more coronary artery bypass or stent. In certain embodiments of the present invention, the patient has a history of one or more venous thromboembolic event or pulmonary embolism. In certain embodiments of the present invention, the patient has a history of one or more non-fatal stroke. In certain embodiments of the present invention, the patient has a history of one or more non-fatal myocardial infarction.

In certain embodiments of the present invention, the patient has a measured ACR≥30 mg/g and ≤300 mg/g (i.e. patient is diagnosed with microalbuminuria. In certain embodiments of the present invention, the patient has a measured ACR>300 mg/g (i.e. patient is diagnosed with macroalbuminuria).

In certain embodiments of the present invention, the patient in need thereof is a patient diagnosed with Type II diabetes mellitus; is further diagnosed with microalbuminuria (ACR≥30 mg/g and ≤300 mg/g) or macroalbuminuria (ACR>300 mg/g); and further exhibits symptoms of or is diagnosed with one or more concomitant or comorbid cardiovascular risk factors or cardiovascular disease.

In certain embodiments of the present invention, the patient has Type II diabetes mellitus and further has one or more of the following characteristics at the time of treatment: a) existing cardiovascular disease or a high likelihood of cardiovascular disease; b) congestive heart failure; c) family history of cardiovascular disease; d) current smoker; e) genetically predisposed to cardiovascular diseases; f) has or has had cardiac arrhythmia; g) has or has had atrial fibrillation, ventricular fibrillation, or tachyarrhythmia; h) does not have sinus tachycardia; i) has unstable angina; j) has hypertension; k) has had a stroke or is at increased risk of stroke; l) has an aneurysm; and/or m) has elevated triglycerides, elevated LDL, and/or low HDL.

In certain embodiments of the present invention, the patient has either a confirmed diagnosis of cardiovascular disease or a high likelihood of cardiovascular disease, and further, said patient has at least one of: a) a history of documented myocardial infarction; b) a history of coronary revascularization; c) a history of carotid or peripheral revascularization; d) angina with ischemic changes; e) ECG changes on a graded exercise test; f) positive cardiac imaging study; g) ankle brachial index<0.9; and/or h) >50% stenosis of a coronary artery, carotid artery, or lower extremity artery.

In certain embodiments of the present invention, the patient has had one or more of the following: (a) a myocardial infarction; (b) a history of angina pectoris; (c) a history of cerebrovascular disease; (d) a history of stroke; (e) a history of tachycardia other than sinus tachycardia; or (f) a planned bariatric surgery, cardiac surgery, or coronary angioplasty.

In certain embodiments, the methods described herein reduce the risk of major adverse cardiovascular events (MACEs).

In certain embodiments of the present invention, the major adverse cardiovascular event is cardiovascular death, non-fatal myocardial infarction, cardiac arrhythmia, or non-fatal stroke. In certain embodiments of the present invention, the major adverse cardiovascular event is cardiovascular death. In certain embodiments of the present invention, the cardiovascular death results from fatal myocardial infarction and/or stroke. In certain embodiments of the present invention, the major adverse cardiovascular event is non-fatal stroke.

In certain embodiments of the present invention, the major adverse cardiovascular event is non-fatal myocardial infarction.

In certain embodiments of the present invention, the methods reduce the predicted severity of an adverse cardiovascular event. In certain embodiments of the present invention, the methods decrease the predicted mortality from an adverse cardiovascular event. In certain embodiments of the present invention, the methods increase the predicted life expectancy of the subject. In certain embodiments of the present invention, the methods increase the predicted time period between adverse cardiovascular events. In certain embodiments of the present invention, the methods increase the effectiveness of a cardiovascular intervention in the subject. In certain embodiments of the present invention, the methods favorably modulate a diagnostic indicator predictive of a major adverse cardiovascular event. In certain embodiments of the present invention, the methods decrease the progression of cardiovascular disease.

In certain embodiments of the present invention, the adverse outcome is one or more events selected from the group consisting of: MACE (including CV death, nonfatal MI, stroke, fatal stroke, nonfatal stroke), nonfatal HUSA (hospitalization due to unstable angina), coronary revascularization procedure, and/or all-cause mortality.

In certain embodiments of the present invention, the methods increase the time until first incidence of one or more events selected from the group consisting of: MACE (including CV death, nonfatal MI, stroke, fatal stroke, nonfatal stroke), nonfatal HUSA (hospitalization due to unstable angina), coronary revascularization procedure, and/or all-cause mortality.

In certain embodiments, the methods of the present invention reduce the predicted severity of an adverse cardiovascular event or decrease the predicted mortality from an adverse cardiovascular event, or decrease the progression of cardiovascular disease.

In certain embodiments, the methods of the present invention increase the predicted life expectancy of the subject, the predicted time period between adverse cardiovascular events, or the effectiveness of a cardiovascular intervention in the subject.

In certain embodiments of the present invention, the methods reduce at least one of: the risk of one or more major adverse cardiovascular events (MACE) in a subject; the predicted severity of an adverse cardiovascular event; the predicted mortality from an adverse cardiovascular event, and combinations thereof, wherein the reduction in risk, predicted severity or predicted mortality is a reduction of at least or at least about 2%, 3%, 5%, 8%, 10%, 12%, 15%, 20%, 25%, 30%, 35%, 40%, 45% or 50%, relative to a subject at the same level of risk of MACE, predicted severity of an adverse cardiovascular event or predicted mortality from an adverse cardiovascular event, but who is not receiving treatment by administration of canagliflozin.

In certain embodiments of the present invention, the methods are effective to decrease the progression of cardiovascular disease in a patient, wherein the decrease in the progression of cardiovascular disease is a decrease of at least or at least about 2%, 3%, 5%, 8%, 10%, 12%, 15%, 20%, 25%, 30%, 35%, 40%, 45% or 50%, in the progression of cardiovascular disease, relative to a patient at the same level of cardiovascular disease progression, but who is not receiving treatment by administration of canagliflozin.

In certain embodiments of the present invention, the methods are effective in increasing the predicted life expectancy of a patient, or in increasing the predicted time period until next occurrence of an adverse cardiovascular event, wherein the increase is at least or at least about 1 month, 2 months, 3 months, 4 months, 5 months, 6 months, 7 months, 8 months, 9 months, 10 months, 11 months, 12 months, 14 months, 16 months, 18 months, 20 months, or 24 months, relative to a patient at the same level of risk of MACE, but who is not receiving treatment by administration of canagliflozin.

In certain embodiments of the present invention, the methods increase the effectiveness of a cardiovascular intervention in a patient, wherein the increase is at least or at least about at least or at least about 2%, 3%, 5%, 8%, 10%, 12%, 15%, 20%, 25%, 30%, 35%, 40%, 45% or 50%, relative to the expected effectiveness of a cardiovascular intervention in a patient at the same level of risk of MACE receiving the same cardiovascular intervention, but who is not receiving treatment by administration of canagliflozin.

In certain embodiments of the present invention, the methods favorably modulate a diagnostic indicator predictive of a major adverse cardiovascular event, wherein the favorable modulation is of at least or at least about at least or at least about 2%, 3%, 5%, 8%, 10%, 12%, 15%, 20%, 25%, 30%, 35%, 40%, 45% or 50%, relative to the diagnostic indicator predictive of a major adverse cardiovascular event in a patient at the same level of risk of MACE, but who is not receiving treatment by administration of canagliflozin.

Canagliflozin may be administered in any composition and according to any dosage regimen established in the art whenever treatment or prevention as described herein is required.

Optimal dosages (of canagliflozin) to be administered may be readily determined by those skilled in the art, and will vary with for example, the mode of administration, the strength of the preparation, and the advancement of the disease condition. In addition, factors associated with the particular patient being treated, including patient age, weight, diet and time of administration, will result in the need to adjust dosages.

In certain embodiments, the present invention is directed to methods for treating or preventing cardiovascular events, wherein canagliflozin is administered at a dosage amount in the range of from about 25 mg to about 500 mg, preferably selected from the group consisting of about 50 mg, about 75 mg, about 100 mg, about 150 mg, about 200 mg, about 300 mg and about 500 mg.

### Definitions

As used herein, unless otherwise noted, the term “canagliflozin” shall mean a compound of formula (I-X)

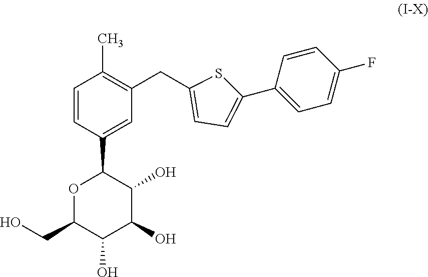 [SMILES: Cc1ccc([C@@H]2O[C@H](CO)[C@@H](O)[C@H](O)[C@H]2O)cc1Cc1ccc(-c2ccc(F)cc2)s1]

or a crystalline hemihydrate form of the compound of formula (I-X).

The compound of formula (I-X) exhibits inhibitory activity against sodium-dependent glucose transporter, such as for example SGLT2; and may be prepared according to the process as disclosed in Nomura, S. et al., US Patent Publication, US 2005/0233988 A1, published Oct. 20, 2005, which is incorporated by reference herein.

As used herein, the term “canagliflozin” shall further include a mixture of stereoisomers, or each pure or substantially pure isomer. In addition, the term “canagliflozin” shall include an intramolecular salt, hydrate, solvate or polymorph thereof.

In an embodiment, the term “canagliflozin” shall mean the crystalline hemihydrate form of the compound of formula (I-X), as described in WO 2008/069327, the disclosure of which is hereby incorporated by reference in its entirety.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder.

As used herein, unless otherwise noted, the terms “delaying the progression of” and “slowing the progression of” shall include (a) delaying or slowing the development of one or more symptoms or complications of the disease, condition or disorder; (b) delaying or slowing the development of one or more new/additional symptoms or complications of the disease, condition or disorder; and/or (c) delaying or slowing the progression of the disease, condition or disorder to a later stage or more serious form of said disease, condition or disorder.

As used herein, unless otherwise noted, the terms “preventing”, “prevention” and the like, shall include (a) reducing the frequency of one or more symptoms; (b) reducing the severity of one or more symptoms; (c) delaying or avoiding of the development of additional symptoms; and/or (d) delaying or avoiding the development of the disorder or condition.

One skilled in the art will recognize that wherein the present invention is directed to methods of prevention, a subject in need of thereof (i.e. a subject or patient in need of prevention) shall include any subject or patient (preferably a mammal, more preferably a human) who has experienced or exhibited at least one symptom of the disorder, disease or condition to be prevented. Further, a subject or patient in need thereof may additionally be a subject or patient (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject or patient may be deemed at risk of developing a disorder, disease or condition (and therefore in need of prevention or preventive treatment) as a consequence of the subject's medical history, including, but not limited to, family history, predisposition, co-existing (comorbid) disorders or conditions, genetic testing, and the like.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “euglycemia” is defined as the condition in which a subject has a fasting blood glucose concentration within the normal range, greater than 70 mg/dL (3.89 mmol/L) and less than 100 mg/dL (5.6 mmol/L), and a 2 h postprandial glucose concentration less than 140 mg/dl.

The term “hyperglycemia” is defined as the condition in which a subject has a fasting blood glucose concentration above the normal range, greater than 100 mg/dL (5.6 mmol/L).

The term “hypoglycemia” is defined as the condition in which a subject has a blood glucose concentration below the normal range, in particular below 70 mg/dL (3.89 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/l. A subject with “normal fasting glucose” has a fasting glucose concentration smaller than 100 mg/dl, i.e. smaller than 5.6 mmol/1.

The term “impaired glucose tolerance” or “GT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio<1.0 (for men) or <0.8 (for women).

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

As a rule, other parameters are used in everyday clinical practice to assess insulin resistance. Preferably, the patient's triglyceride concentration is used, for example, as increased triglyceride levels correlate significantly with the presence of insulin resistance.

Patients with a predisposition for the development of IGT or IFG or Type 2 diabetes are those having euglycemia with hyperinsulinemia and are by definition, insulin resistant. A typical patient with insulin resistance is usually overweight or obese. If insulin resistance can be detected, this is a particularly strong indication of the presence of pre-diabetes. Thus, it may be that in order to maintain glucose homoeostasis a person needs 2-3 times as much insulin as a healthy person, without this resulting in any clinical symptoms.

The term “pre-diabetes” is the condition wherein an individual is predisposed to the development of type 2 diabetes. Pre-diabetes extends the definition of impaired glucose tolerance to include individuals with a fasting blood glucose within the high normal range 100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749). Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes.

The terms “Type 2 diabetes” and “Type II diabetes mellitus” are defined as the condition in which a subject has a fasting (i.e., no caloric intake for 8 hours) blood glucose or serum glucose concentration greater than 125 mg/dL (6.94 mmol/L), when measured at minimum two independent occasions. The measurement of blood glucose values is a standard procedure in routine medical analysis. Type 2 diabetes is also defined as the condition in which a subject has HbA1c equal to, or greater than 6.5%, a two hour plasma glucose equal to, or greater than 200 mg/dL (11.1 mmol/L) during an oral glucose tolerance test (OGTT) or a random glucose concentration equal to, or greater than 200 mg/dL (11.1 mmol/L) in conjunction with classic symptoms of hyperglycaemia or hyperglycaemic crisis. In the absence of unequicoval hyperglycaemia, as with most diagnostic tests, a test result diagnostic of diabetes should be repeated to rule out laboratory error. The assessment of HbA1c should be performed using a method certified by the National Glycohemoglobin Standardization Program (NGSP) and standardized or traceable to the Diabetes Control and Complications Trial (DCCT) reference assay. If a OGTT is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after a minimum of 8 hours, typically after 10-12 hours, of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage Type 2 diabetes mellitus” includes patients with a long-standing duration of diabetes, secondary drug failure, indication for insulin therapy and potentially progression to micro- and macrovascular complications e.g. diabetic nephropathy, or coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <6.5% and preferably <6% HbA1c.

The terms “metabolic syndrome”, “syndrome X” (when used in the context of a metabolic disorder), and “dysmetabolic syndrome” refer to a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference greater than about 40 inches or 102 cm in men, and greater than about 35 inches or 94 cm in women;
2. Triglycerides equal to or greater than about 150 mg/dL;
3. HDL-cholesterol less than about 40 mg/dL in men and less than about 50 in women;
4. Blood pressure equal to or greater than about 130/85 mm Hg (SBP equal to or greater than about 130 or DBP equal to or greater than about 85);
5. Fasting blood glucose equal to or greater than about 100 mg/dL.

It is intended that patients diagnosed with Metabolic Syndrome or Syndrome X are included within the methods of the present invention.

The term “body mass index” or “BMI” of a human patient is defined as the weight in kilograms divided by the square of the height in meters, such that BMI has units of kg/m². The term “overweight” is defined as the condition wherein the adult individual of Europid origin has a BMI equal to or greater than 25 kg/m² and less than 30 kg/m². In subjects of Asian origin the term “overweight” is defined as the condition wherein the adult individual has a BMI equal to or greater than 23 kg/m² and less than 25 kg/m². The terms “overweight” and “pre-obese” are used interchangeably.

The term “obesity” is defined as the condition wherein the adult individual of Europid origin has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the terms “class III obesity” is the condition wherein the BMI is equal to or greater than 40 kg/m². In subjects of Asian origin the term “obesity” is defined as the condition wherein the adult individual has a BMI equal or greater than 25 kg/m². Obesity in Asians may be categorized further as follows: the term “class I obesity” is the condition wherein the BMI is equal to or greater than 25 kg/m² but lower than 30 kg/m²; the term “class II obesity” is the condition wherein the BMI is equal to or greater than 30 kg/m².

The term “visceral obesity” is defined as the condition wherein a waist-to-hip ratio of greater than or equal to 1.0 in men and 0.8 in women is measured. It defines the risk for insulin resistance and the development of pre-diabetes. The term “abdominal obesity” is usually defined as the condition wherein the waist circumference is >40 inches or 102 cm in men, and is >35 inches or 94 cm in women (for normal ranges of populations, see for example “Joint scientific statement (IDF, NHLBI, AHA, WHO, IAS, IASO). Circulation 2009; 120:1640-1645”).

The term “morbid obesity” is defined herein as a condition in which the individual of Europid origin has a BMI>40 or has a BMI>35 and a comorbidity such as diabetes mellitus or hypertension (see World Health Organization. Obesity: Preventing and Managing the Global Epidemic: Report on a WHO Consultation. World Health Organ Tech Rep Ser. 2000; 894: i-xii, 1-253).

According to a commonly used definition, hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

As used herein, unless otherwise noted, the term “cardiovascular risk factors” includes, but is not limited to hypertension or high blood pressure (for example, elevated systolic blood pressure, elevated diastolic blood pressure, or a blood pressure of greater than about 145/95 mm Hg), elevated cholesterol (hyperlipidemia), elevated LDL, depressed HDL levels, elevated triglycerides, obesity (as defined by for example, a BMI of greater than 30, preferably, morbid obesity defined by a BMI of greater than 40), cardiovascular disease (including, but not limited to, previous myocardial infarction, angina, heart failure, stroke), microalbuminuria (as defined for example by ACR≥30 mg/g and ≤300 mg/g), macroalbuminuria (as defined by for example ACR>300 mg/g), peripheral vascular disease (including, but not limited to, carotid stenosis, femoral artery stenosis, and the like), underlying structural heart disease, atrial fibrillation, tachycardia, coronary disease, non-rheumatic heart valve disease, dilated cardiomyopathy of ischemic origin, ablation of atrial fibrillation or flutter, (including, but not limited to catheter ablation or endomyocardial ablation), supraventricular tachycardia other than atrial fibrillation or flutter, history of heart valve surgery, non-ischemic dilated cardiomyopathy, hypertrophic cardiomyopathy, rheumatic valve disease, sustained ventricular tachycardia, congenital cardiopathy, ablation (including but not limited to catheter ablation, for tachycardia other than for atrial fibrillation or flutter), ventricular fibrillation, at least one cardiac device (including, but not limited to a cardiac stimulator, an implantable defibrillator (“ICD”), and the like), current or past history of smoking and male gender.

In certain embodiments, the cardiovascular risk factors include hypertension or high blood pressure (for example, elevated systolic blood pressure, elevated diastolic blood pressure, or a blood pressure of greater than about 145/95 mm Hg), elevated cholesterol (hyperlipidemia), elevated LDL, depressed HDL levels, elevated triglycerides, obesity (as defined by for example, a BMI of greater than 30, preferably, morbid obesity defined by a BMI of greater than 40), cardiovascular disease (including, but not limited to, previous myocardial infarction, angina, heart failure, stroke), microalbuminuria (as defined for example by ACR≥30 mg/g and ≤300 mg/g) and macroalbuminuria (as defined by for example ACR>300 mg/g).

In certain embodiments, the one or more cardiovascular risk factor(s) are selected from those identified in the patient population which completed the CANVAS or CANVAS-R clinical trial detailed herein.

As used herein, unless otherwise noted, the term “reducing cardiovascular risk” shall include reducing the symptoms or hallmarks of cardiovascular disease, halting or slowing the progression of cardiovascular disease, and/or halting, slowing the progression or controlling any one or more of the risk factors associated with cardiovascular disease.

As used herein, unless otherwise noted, the term “cardiovascular disease” shall include, but is not limited to, history of non-fatal myocardial infarction, history of non-fatal stroke (ischemia), peripheral artery disease, hypertensive heart disease, ischemic heart disease, coronary vascular disease, peripheral vascular disease, cerebrovascular disease, cardiac arrhythmia (other than sinus tachychardia), cardiomyopathy, angina (including but not limited to unstable angina), heart failure (including, but not limited to heart failure requiring hospitalization, congestive heart failure, and the like) and coronary valve disease.

In certain embodiments of the present invention, the one or more cardiovascular disease(s) are selected from those identified in the patient population which completed the CANVAS or CANVAS-R clinical trial detailed herein.

As used herein, unless otherwise noted, the term “major adverse cardiovascular events (“MACEs”)” shall include three primary measurements: nonfatal myocardial infarction (“MI”), nonfatal stroke, and cardiovascular death. One skilled in the art will recognize that these major adverse cardiovascular events represent serious ischemic events and are widely used endpoints in cardiovascular outcome trials.

In certain embodiments of the present invention, the major adverse cardiovascular event is cardiovascular death. In certain embodiments of the present invention, the cardiovascular death comprises death resulting from fatal myocardial infarction and/or fatal stroke.

In certain embodiments of the present invention, the major adverse cardiovascular event is non-fatal stroke. In certain embodiments of the present invention, the major adverse cardiovascular event is non-fatal myocardial infarction. In certain embodiments of the present invention, the major adverse cardiovascular event is cardiac arrhythmia. In certain embodiments of the present invention, the major adverse cardiovascular event further comprises progression from unstable angina to myocardial infarction or death.

As used herein, unless otherwise noted, the term “cardiovascular event” shall include, but is not limited to, cardiovascular hospitalization, non-fatal myocardial infarction, non-fatal ischemia or stroke, and cardiovascular mortality.

As used herein, unless otherwise noted, the term “reducing the risk of a cardiovascular event” shall include one or more of the following: reducing the risk of a non-fatal myocardial infarction, reducing the risk of non-fatal ischemic event or stroke, reducing the risk of hospitalization due to one or more cardiac symptoms or events, or reducing the risk of cardiovascular mortality.

The term “cardiovascular hospitalization” means a hospitalization which is caused by at least one of the following pathologies (Hohnloser et al., Journal of cardiovascular electrophysiology, January 2008, vol. 19, No. 1, pages 69-73):

atherosclerosis, myocardial infarction or unstable angina pectoris, stable angina pectoris or atypical thoracic pain, syncope, transient ischemic event or cerebral stroke (except intracranial haemorrhage), atrial fibrillation and other supraventricular rhythm disorders, non-fatal cardiac arrest, ventricular arrhythmia, cardiovascular surgery, except heart transplant, heart transplant, implantation of a cardiac stimulator (pacemaker), of an implantable defibrillator (“ICD”) or of another cardiac device, percutaneous coronary, cerebrovascular or peripheral intervention, variations in arterial pressure (hypotension, hypertension, except syncope), cardiovascular infection, major bleeding/haemorrhage (requiring two or more blood cell pellets or any intracranial haemorrhage), pulmonary embolism or deep vein thrombosis, worsening of congestive heart failure including acute pulmonary oedema or dyspnoea from cardiac causes. Prevention of cardiovascular hospitalization shall further include prevention of hospitalizations for transient ischemic event, cardiovascular ischemia or cerebral stroke.

In the methods of the present invention, the prevention of cardiovascular hospitalization may be understood as the prevention of cardiovascular hospitalization for any one or more of the above mentioned pathologies.

As used herein, unless otherwise noted, the term “mortality” or “death” are equivalent and includes mortality due to any cause, whether cardiovascular or non-cardiovascular or unknown.

As used herein, unless otherwise noted, the term “cardiovascular mortality” includes mortality due to any cardiovascular cause (any death except those due to a non-cardiovascular cause), including mortality due to, for example:
a) Aortic dissection/aneurysm;
b) Cardiac tamponade;
c) Cardiogenic shock;
d) Congestive heart failure;
e) Death during a cardiovascular transcutaneous interventional procedure or cardiovascular surgical intervention;
f) Myocardial infarction or unstable angina (including complications of myocardial infarction, except arrhythmias);
g) Pulmonary or peripheral embolism;
h) Stroke (ischemia);
i) Sudden cardiac death (e.g., unwitnessed death or documented asystole);
j) Ventricular arrhythmia, subclassified as torsades de pointes, ventricular extrasystole, ventricular fibrillation, ventricular tachycardia (non-sustained and sustained ventricular tachycardia), or other ventricular arrhythmia; and
k) Unknown cause.

As used herein, unless otherwise noted, the term “sudden death” refers, in general, to death occurring within the hour or less than one hour after the appearance of new symptoms or unexpected death without warning.

As used herein, unless otherwise noted, the term “coronary disease” or “coronary heart disease” refers to:
a) Coronary artery disease: documented history of acute myocardial infarction and/or significant (˜70%) coronary artery stenosis and/or history of a revascularization procedure (percutaneous transluminal coronary angioplasty, stent implantation in a coronary artery, coronary artery bypass graft, etc) and/or a positive exercise test and/or positive nuclear scan of cardiac perfusion; and
b) Ischemic dilated cardiomyopathy: clinically significant left ventricular dilatation secondary to coronary artery disease.

One skilled in the art will recognize that “prevention of cardiovascular hospitalization and/or mortality” results in the reduction of the risk of cardiovascular hospitalization and or mortality or in the reduction of the need of cardiovascular hospitalization and or mortality.

As used herein, unless otherwise noted, the term “structural heart disease” shall include coronary heart disease and/or ischemic dilated cardiomyopathy and/or non-ischemic dilated cardiomyopathy and/or rheumatic valvular heart disease and/or non-rheumatic valvular heart disease and/or hypertrophic cardiomyopathy and/or LVEF<45% and/or history of congestive heart failure wherein congestive heart failure may be defined for example as NYHA (New York Heart Association) class III or by a reduced left ventricular ejection fraction below 0.35.

As used herein, unless otherwise noted, the term “renal disorders” shall mean any disorder related to or affecting kidney function and/or renal hyperfiltration. Renal disorders including, but are not limited to elevated urine albumin level, elevated serum albumin/creatinine ratio, microalbuminuria, macroalbuminuria, renal hyperfiltrative injury, diabetic nephropathy (including, but not limited to hyperfiltrative diabetic nephropathy), renal hyperfiltration, glomerular hyperfiltration, renal allograft hyperfiltration, compensatory hyperfiltration, hyperfiltrative chronic kidney disease, hyperfiltrative acute renal failure, and obesity.

According to the National Kidney Foundation (NKF) Kidney Disease Outcomes Quality Initiative (KDOQI), Guidelines for Screening and Diagnosis of Diabetic Kidney Disease, microalbuminuria is diagnosed in a subject (patient) whose albumin-creatinine ratio (ACR) is between 30 mg/g and 300 mg/g; and macroalbuminuria is diagnosed in a subject (patient) whose albumin-creatinine ration (ACR) is greater than 300 mg/g.

The term “hyperfiltration” is defined as an elevation in the filtration rate of the renal glomeruli. In one aspect, hyperfiltration is defined as a whole kidney filtration rate equal to or greater than about 125 mL/min/1.73 m², especially equal to or greater than about 140 mL/min/1.73 m², as measured using a method described herein below. Hyperfiltration may also be defined as related to an absolute GFR greater to the about 90^{th}, or the about 95^{th} percentile in the studied population after adjusting for sex, age, weight, height, and the use of ACE inhibitors or ARB (Melsom et al. Diabetes Care 2011; DOI: 10.2337/dc11-0235).

The term “glomerular filtration rate (GFR)” is defined as the volume of fluid filtered from the renal (kidney) glomerular capillaries into the Bowman's capsule per unit time. It is indicative of overall kidney function. The glomerular filtration rate (GFR) may be calculated by measuring any chemical that has a steady level in the blood, and is freely filtered but neither reabsorbed nor secreted by the kidneys. The rate therefore measured is the quantity of the substance in the urine that originated from a calculable volume of blood. The GFR is typically recorded in units of volume per time, e.g., milliliters per minute and the formula below can be used:

$$G F R = \frac{( Urine Concentration \times Urine Volume )}{Plasma Concentration}$$

The GFR may be determined by injecting inulin into the plasma. Since inulin is neither reabsorbed nor secreted by the kidney after glomerular filtration, its rate of excretion is directly proportional to the rate of filtration of water and solutes across the glomerular filter. A normal value is: GFR=90-125 mL/min/1.73 m², in particular GFR=100-125 mL/min/1.73 m². Other principles to determine GFR involve measuring 51Cr-EDTA, [125I]iothalamate or iohexyl.

The “estimated glomerular filtration rate (eGFR)” is defined as derived at screening from serum creatinine values based on e.g., the Chronic Kidney Disease Epidemiology Collaboration (CKD-EPI) equation, the Cockcroft-Gault formula or the Modification of Diet in Renal Disease (MDRD) formula, which are all known in the art. Subjects with normal renal function are defined as eGFR equal to or greater than 90 ml/min. Subjects with mild impairment of renal function as defined eGFR equal to or greater than 60 and less than 90 ml/min). Subjects with moderate impairment as defined as eGFR equal to or greater than 30 and less than 60 ml/min). Subjects with severe impairment as defined as eGFR equal to or greater than 15 and less than 30 ml/min.

The term “renal hyperfiltrative injury” is defined as a manifestation of renal damage caused predominantly by renal hyperfiltration, which often is an early link in the chain of events to further renal injury, acknowledging that hyperfiltration often works in concert with other chronic kidney disease risk factors in the pathogenesis of renal injury.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. Further, to provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Pharmaceutical compositions canagliflozin as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like, for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare such pharmaceutical compositions, canagliflozin, as the active ingredient, is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, from about 25 mg to about 500 mg of canagliflozin or any amount or range therein (preferably selected from the group consisting of about 50 mg, about 75 mg, about 100 mg, about 150 mg, about 200 mg, and about 300 mg of canagliflozin. The dosages, however, may be varied depending upon the requirement of the patients, the severity of the condition being treated and the compound being employed. The use of either daily administration or post-periodic dosing may be employed.

Preferably the pharmaceutical compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. For preparing solid compositions such as tablets, the principal active ingredient (e.g. canagliflozin) is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. In certain embodiments, two active ingredients can be formulated together, e.g., in a bi-layer tablet formulation. When referring to these preformulation compositions as homogeneous, it is meant that the active ingredients are dispersed evenly throughout the composition so that the composition may be readily subdivided into equally effective dosage forms such as tablets, pills and capsules. This solid preformulation composition is then subdivided into unit dosage forms of the type described above containing from about 25 mg to about 500 mg of canagliflozin or any amount or range therein. The tablets or pills of the composition can be coated or otherwise compounded to provide a dosage form affording the advantage of prolonged action. For example, the tablet or pill can comprise an inner dosage and an outer dosage component, the latter being in the form of an envelope over the former.

The liquid forms in which the compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described herein may also be carried out using a pharmaceutical composition comprising canagliflozin and a pharmaceutically acceptable carrier. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, canagliflozin may be administered in a single daily dose, or the total daily dosage may be administered in divided doses of two, three or four times daily.

For instance, for oral administration in the form of a tablet or capsule, the active drug component (e.g. canagliflozin) can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methylcellulose and the like. For parenteral administration, sterile suspensions and solutions are desired. Isotonic preparations which generally contain suitable preservatives are employed when intravenous administration is desired.

To prepare a pharmaceutical composition of the present invention, canagliflozin, as the active ingredient, may be intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain, the disclosure of which is hereby incorporated by reference.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc., the disclosures of which are hereby incorporated by reference.

The following Examples are set forth to aid in the understanding of the invention, and are not intended and should not be construed to limit in any way the invention set forth in the claims which follow thereafter.

In terms of the clinical studies detailed below, in certain instances (for example in the primary endpoint), the prevention of “cardiovascular events or cardiovascular mortality” constitute what are referred to as composite criteria or a combined endpoint.

### Example 1

### CANVAS and CANVAS-R Clinical Trials

Two clinical trials were completed to assess the cardiovascular safety and efficacy of canagliflozin, and how any potential benefits might balance against known risks. The complete protocols for said trials, name CANVAS and CANVAS-R, which protocols are each incorporated in their entirety herein, may be found on www.clinicaltrials.gov (more specifically at the following urls: https://clinicaltrials.gov/ct2/show/NCT01032629?term=canvas&rank=1 and https://clinicaltrials.gov/ct2/show/NCT01989754?term=canvas-r&rank=1, respectively).

### Participants

Participants were men and women with type 2 diabetes (glycated hemoglobin≥7.0% and ≤10.5%) either 30 years or older with a history of symptomatic atherosclerotic cardiovascular disease, or 50 years or older with two or more of the following risk factors for cardiovascular disease: duration of diabetes≥10 years, systolic blood pressure>140 mmHg while on one or more antihypertensive agents, current smoker, microalbuminuria or macroalbuminuria, or high-density lipoprotein (HDL) cholesterol<1 mmol/L. Participants were required to have an estimated glomerular filtration rate at entry of >30 ml/min/1.73 m² and to the criteria listed in Table 1 below.

TABLE 1
Inclusion and Exclusion Criteria for Patent Participation

| | |
|---|---|
| CANVAS | CANVAS-R |

INCLUSION CRITERIA

| | |
|---|---|
| Man or woman with a diagnosis of type 2 | Same |
| diabetes with glycated hemoglobin level ≥7.0% | |
| to ≤10.5% at screening and be either (1) not | |
| currently on antihyperglycemic agent (AHA) | |
| therapy or (2) on AHA monotherapy or | |
| combination therapy with any approved class | |
| of agents: eg, sulfonylurea, metformin, | |
| peroxisome proliferator-activated receptor | |
| gamma (PPARγ) agonist, alpha-glucosidase | |
| inhibitor, glucagon-like peptide-1 (GLP-1) | |
| analogue, dipeptidyl peptidase-4 (DPP-4) | |
| inhibitor, or insulin. | |
| History or high risk of cardiovascular (CV) | Same |
| disease defined on the basis of either: | |
| Age ≥30 years with documented symptomatic | |
| atherosclerotic CV disease: including stroke; | |
| myocardial infarction (MI), hospital admission | |
| for unstable angina; coronary artery bypass | |
| graft; percutaneous coronary intervention (with | |
| or without stenting); peripheral | |
| revascularization (angioplasty or surgery); | |
| symptomatic with documented | |
| hemodynamically-significant carotid or | |
| peripheral vascular disease; or amputation | |
| secondary to vascular disease | |
| Age ≥50 years with 2 or more of the following | |
| risk factors determined at the screening visit: | |
| duration of type 2 diabetes of 10 years or more, | |
| systolic blood pressure >140 mmHg (average | |
| of 3 readings) recorded at the Screening Visit, | |
| while the subject is on at least one blood | |
| pressure-lowering treatment, current daily | |
| cigarette smoker, documented | |
| microalbuminuria or macroalbuminuria, or | |
| documented high-density lipoprotein (HDL) | |
| cholesterol of <1 mmol/l (<39 mg/dl). | |
| Women must be: | Same |
| Postmenopausal, defined as >45 years of | |
| age with amenorrhea for at least 18 months, or | |
| >45 years of age with amenorrhea for at least 6 | |
| months and less than 18 months and a serum | |
| follicle stimulating hormone (FSH) level >40 | |
| IU/ml, or | |
| Surgically sterile (have had a hysterectomy or | |
| bilateral oophorectomy, tubal ligation), or | |
| otherwise be incapable of pregnancy, or | |
| Heterosexually active and practicing a highly | |
| effective method of birth control, including | |
| hormonal prescription oral contraceptives, | |
| contraceptive injections, contraceptive patch, | |
| intrauterine device, double-barrier method (eg, | |
| condoms, diaphragm, or cervical cap with | |
| spermicidal foam, cream, or gel), or male | |
| partner sterilization, consistent with local | |
| regulations regarding use of birth control | |
| methods for subjects participating in clinical | |
| trials, for the duration of their participation in | |
| the study, or | |
| not heterosexually active. | |
| Note: subjects who are not heterosexually | |
| active at screening must agree to utilize a | |
| highly effective method of birth control if they | |
| become heterosexually active during their | |
| participation in the study. | |
| Women of childbearing potential must have a | Same |
| negative urine β-human chorionic gonadotropin | |
| (β-hCG) pregnancy test at screening and | |
| baseline (predose, Day 1). | |
| Willing and able to adhere to the prohibitions | Same |
| and restrictions specified in this protocol. | |
| Subjects must have signed an informed | |
| consent document indicating that they | |
| understand the purpose of and procedures | |
| required for the study and are willing to | |
| participate in the study. | |
| To participate in the optional | N/A |
| pharmacogenomic component of this study, | |
| subjects must have signed the informed | |
| consent form for pharmacogenomic research | |
| indicating willingness to participate in the | |
| pharmacogenomic component of the study | |
| (where local regulations permit). Refusal to | |
| give consent for this component does not | |
| exclude a subject from participation in the | |
| clinical study. | |
| Subjects must have taken ≥80% of their single- | Same |
| blind placebo capsules during the 2-week run- | |
| in period at Day 1 to be eligible for | |
| randomization. | |

EXCLUSION CRITERIA

| | |
|---|---|
| History of diabetic ketoacidosis, type 1 | Same |
| diabetes, pancreas or beta-cell transplantation, | |
| or diabetes secondary to pancreatitis or | |
| pancreatectomy. | |
| On an AHA and not on a stable regimen (i.e., | N/A |
| agents and doses) for at least 8 weeks before | |
| the screening visit and through the | |
| screening/run-in period. Note: a stable dose of | |
| insulin is defined as no change in the insulin | |
| regimen (i.e., type[s] of insulin) and ≤15% | |
| change in the total daily dose of insulin | |
| (averaged over 1 week to account for day-to- | |
| day variability). | |
| Fasting fingerstick glucose at site >270 mg/dl | Not included |
| (>15 mmol/l) at Baseline/Day 1 | |
| For patients on a sulfonylurea agent or on | |
| insulin: fasting fingerstick glucose at site <110 | |
| mg/dl (<6 mmol/l) at Baseline/Day 1 | |
| Note: at the investigator's discretion, based | |
| upon an assessment of recent self-monitored | |
| blood glucose (SMBG) values, subjects | |
| meeting either of these fingerstick glucose | |
| exclusion criteria may continue the single-blind | |
| placebo and return to the investigational site | |
| within 14 days and may be randomized if the | |
| repeat fasting fingerstick value no longer meets | |
| the exclusion criterion. Subjects with | |
| fingerstick glucose >270 mg/dl (>15 mmol/l) | |
| may have their AHA regimen adjusted, and be | |
| rescreened once on a stable regimen for at | |
| least 8 weeks. | |
| History of one or more severe hypoglycemic | Same |
| episode within 6 months before screening. | |
| Note: a severe hypoglycemic episode is | |
| defined as an event that requires the help of | |
| another person. | |
| History of hereditary glucose-galactose | Same |
| malabsorption or primary renal glucosuria. | |
| Ongoing, inadequately controlled thyroid | Same |
| disorder. Note: subjects on thyroid hormone | |
| replacement therapy must be on a stable dose | |
| for at least 6 weeks before Day 1. | |
| Renal disease that required treatment with | Same |
| immunosuppressive therapy or a history of | |
| dialysis or renal transplant. Note: subjects with | |
| a history of treated childhood renal disease, | |
| without sequelae, may participate. | |
| MI, unstable angina, revascularization | same |
| procedure, or cerebrovascular accident within 3 | |
| months before screening, or a planned | |
| revascularization procedure, or history of New | |
| York Heart Association (NYHA) Class IV | |
| cardiac disease. | |
| Findings on 12-lead electrocardiogram (ECG) | Known ECG findings |
| that would require urgent diagnostic evaluation | within 3 months |
| or intervention (e.g., new clinically important | before screening that |
| arrhythmia or conduction disturbance). | would require urgent |
| | diagnostic evaluation |
| | or intervention |
| | (e.g., new clinically |
| | important arrhythmia or |
| | conduction disturbance). |
| History of hepatitis B surface antigen or | Same |
| hepatitis C antibody positive (unless associated | |
| with documented persistently stable/normal | |
| range aspartate aminotransferase [AST] and | |
| alanine aminotransferase [ALT] levels), or other | |
| clinically active liver disease. | |
| Any history of or planned bariatric surgery. | Same |
| Estimated glomerular filtration rate (eGFR) <30 | eGFR <30 ml/min/ |
| ml/min/1.73 m² at screening (provided by the | 1.73 m² at |
| central laboratory) | screening visit. |
| For subjects taking metformin: at screening, | |
| serum creatinine ≥1.4 mg/dl (124 μmol/l) for | |
| men or ≥1.3 mg/dl (115 μmol/l) for women; no | |
| contraindication to the use of metformin | |
| (including eGFR) based on the label of the | |
| country of investigational site | |
| ALT levels >2.0 times the upperlimit of normal | Same |
| (ULN) or total bilirubin >1.5 times the ULN at | |
| screening, unless in the opinion of the | |
| investigator and as agreed upon by the | |
| sponsor's medical officer, the findings are | |
| consistent with Gilbert's disease. | |
| History of malignancy within 5 years before | Same |
| screening (exceptions: squamous and basal | |
| cell carcinomas of the skin and carcinoma of | |
| the cervix in situ, or a malignancy that in the | |
| opinion of the investigator, with concurrence | |
| with the sponsor's medical monitor, is | |
| considered cured with minimal risk of | |
| recurrence). | |
| History of human immunodeficiency | Same |
| virus (HIV) antibody positive. | |
| Subject has a current clinically important | Same |
| hematological disorder (e.g., symptomatic | |
| anemia, proliferative bone marrow disorder, | |
| thrombocytopenia). | |
| Investigator's assessment that the subject's life | Same |
| expectancy is less than 1 year, or any condition | |
| that in the opinion of the investigator would | |
| make participation not in the best interest of the | |
| subject, or could prevent, limit, or confound the | |
| protocol-specified safety or efficacy | |
| assessments. | |
| Major surgery (i.e., requiring general | Same |
| anesthesia) within 3 months of the screening | |
| visit or any surgery planned during the | |
| subject's expected participation in the study | |
| (except minor surgery; i.e., outpatient surgery | |
| under local anesthesia) | |
| Any condition that, in the opinion of the | Same |
| investigator, would compromise the well-being | |
| of the subject or prevent the subject from | |
| meeting or performing study requirements | |
| N/A | Prior or current |
| | participation in another |
| | canagliflozin study. |
| Current use of other sodium glucose co- | Current or prior use of |
| transporter 2 (SGLT2) inhibitor. | an SGLT2 inhibitor. |
| Known allergies, hypersensitivity, or | Same |
| intolerance to canagliflozin or its excipients. | |
| Current use of a corticosteroid medication or | Same |
| immunosuppressive agent, or likely to require | |
| treatment with a corticosteroid medication (for | |
| longer than 2 weeks in duration) or an | |
| immunosuppressive agent. Note: subjects | |
| using inhaled, intranasal, intra-articular, or | |
| topical corticosteroids, or corticosteroids in | |
| therapeutic replacement doses may participate. | |
| Received an active investigational drug | Same |
| (including vaccines) or used an investigational | |
| medical device within 3 months before Day | |
| 1/baseline or received at least one dose of | |
| canagliflozin in a prior study. | |
| History of drug or alcohol abuse within 3 years | Same |
| before screening. | |
| Pregnant or breastfeeding or planning to | Same |
| become pregnant or breastfeed during the | |
| study. | |
| Employees of the investigator or study center, | Same |
| with direct involvement in the proposed study | |
| or other studies under the direction of that | |
| investigator or study center, as well as family | |
| members of the employees or the investigator. | |

### Study Protocol Summary

All potential participants completed a 2-week, single-blind, placebo run-in period. Randomization was done centrally through an interactive web response system using a computer-generated randomization schedule prepared by the study sponsor using randomly permuted blocks. Participants in CANVAS were randomly assigned in a 1:1:1 ratio to canagliflozin 300 mg, canagliflozin 100 mg, or matching placebo, and participants in CANVAS-R were randomly assigned in a 1:1 ratio to canagliflozin or matching placebo administered at an initial dose of 100 mg daily with optional up-titration to 300 mg from week 13. Participants and all study staff were masked to individual treatment allocations until the completion of the study. Use of other background therapy for glycemic management and other risk factor control was according to best practice instituted in line with local guidelines including Renin Angiotensin System (RAS) blockade.

Post-randomization, face-to-face follow-up was scheduled for 3 visits in the first year and at 6-month intervals thereafter, with alternating telephone follow-up between face-to-face assessments. Every follow-up included inquiry about primary and secondary outcome events and serious adverse events. Urinary albumin:creatinine ratio was measured every 26 weeks in CANVAS-R, and at Week 12 and then annually in CANVAS. Serum creatinine measurement with estimation of glomerular filtration rate (eGFR) was performed at least every 26 weeks in both trials. Individuals that prematurely discontinued study treatment continued scheduled follow-up wherever possible, with extensive efforts made to obtain full outcome data for all during the final follow-up window that spanned November 2016 to February 2017.

### Outcomes

The primary outcome was a composite of cardiovascular mortality, nonfatal myocardial infarction, or nonfatal stroke. Secondary outcomes were total mortality; cardiovascular mortality; progression of albuminuria; and the composite of cardiovascular mortality and hospitalization for heart failure. Progression of albuminuria grade was defined as a more then 30% increase in albuminuria and a switch from either normoalbuminuria to microalbuminuria or macroalbuminuria, or from microalbuminuria to macroalbuminuria. In the event that sequential testing was not significant for all then remaining outcomes were scheduled for assessment as exploratory variables in the integrated dataset.

Exploratory cardiovascular outcomes prespecified for evaluation were nonfatal myocardial infarction, nonfatal stroke and hospitalization for heart failure and the key pre-specified exploratory renal endpoints were regression of albuminuria (using comparable criteria to those defined for grade progression) and the renal composite comprising a 40% reduction in eGFR sustained for at least two consecutive measures, the need for renal replacement therapy (dialysis or transplantation) or renal death (defined as a death with a proximate renal cause). Evaluation of total hospitalizations was also prespecified.

All major cardiovascular events, renal outcomes and deaths, plus selected safety outcomes were ratified by Endpoint Adjudication Committees. Intermediate markers of cardiovascular risk and requirement for antihyperglycemic agents were assessed to help understand the observed effects on cardiovascular and renal outcomes. Analyses of safety were of adverse events coded using the latest version of the MedDRA Dictionary. For bone fracture, the primary prespecified analysis was for low-trauma fracture events but secondary analysis of all fractures was also done. Amputations were assessed overall but numbers of cases above and below the ankle were also reported.

Cardiovascular, renal and cause of death criteria applied in the clinical trials (including MACE criteria) were as outlined in detail in the clinical trial protocols, available on www.clinicaltrials.gov, which are incorporated in their entirety herein.

### Statistical Analysis

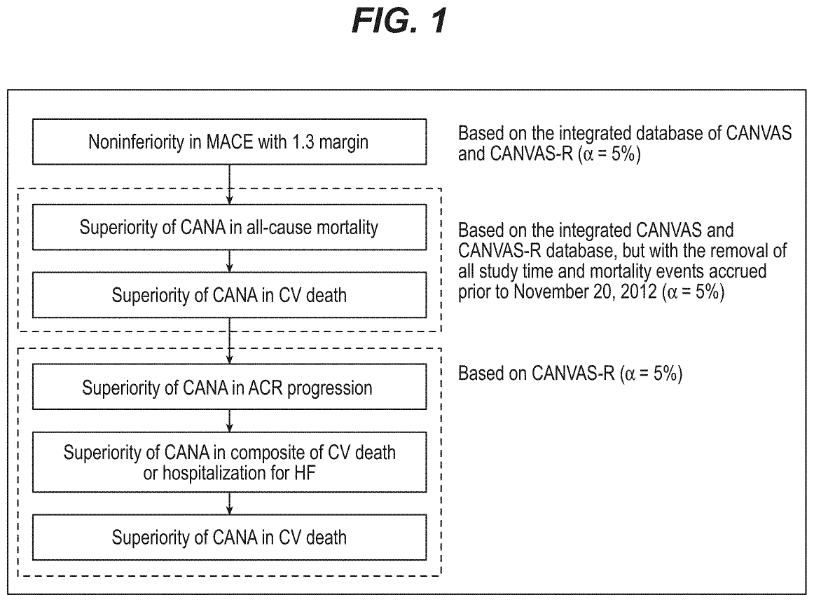

The primary hypothesis test was of noninferiority for the hazard ratio (HR) for the primary outcome at the margin of 1.3 for all canagliflozin versus placebo using the full integrated dataset and the intent to treat approach. Cardiovascular safety was demonstrated if, as compared to placebo, the upper bound of the 95% confidence interval (C) of the HR was less than 1.3 and superiority if the upper bound was also less than 1.0. Hypothesis testing was scheduled to proceed sequentially conditional on the primary safety hypothesis and each subsequent test for superiority being met, as shown in the flowchart in FIG. 1 and based on the values listed in Table 2, below.

TABLE 2
Hazard ratio, 95% CIs and P values for Prespecified Sequential
Hypothesis Testing Plan

| | | | |
|---|---|---|---|
| Canagli- | Placebo | | |
| flozin | Per | Hazard ratio | |
| Per 1000 | 1000 | (95% | |
| patient- | patient- | confidence | |
| years | years | interval) | P value |

Based on the integrated database of CANVAS and CANVAS-R

| | | | | |
|---|---|---|---|---|
| Primary outcome | 26.93 | 31.48 | 0.86 (0.75-0.97) | <0.0001 |
| | | | | * |
| | | | | 0.0158^{†} |

Based on the integrated database of CANVAS and
CANVAS-R, but with the removal of all study time
and mortality events accrued prior to Nov. 20, 2012

| | | | | |
|---|---|---|---|---|
| All-cause mortality | 19.05 | 20.12 | 0.90 (0.76-1.07) | 0.2452 |
| Cardiovascular death | 12.82 | 12.74 | 0.96 (0.77-1.18) | NA |
| Based on CANVAS-R | | | | |
| Albumin:creatinine | 99.80 | 153.01 | 0.64 (0.57-0.73) | NA |
| ratio progression | | | | |
| Cardiovascular death | 15.85 | 21.91 | 0.72 (0.55-0.94) | NA |
| or hospitalization for | | | | |
| heart failure | | | | |
| Cardiovascular death | 10.06 | 11.60 | 0.86 (0.61-1.22) | NA |

* Noninferiority P value.
^{†}Superiority P value.
NA = not applicable because prior P > 0.05

In addition to the formal hypothesis testing, a supplementary set of exploratory analyses of cardiovascular outcomes, renal outcomes, death and hospitalizations was prespecified based on the full integrated dataset comprising all randomized participants. Hazard ratios, 95% CIs, and P values were estimated by using Cox regression models, with stratification by trial and prior history of cardiovascular disease, for all canagliflozin groups combined versus placebo.

P values for efficacy were reported only where the hypothesis was proven. Supplementary analysis using imputation for missing data by multiple imputation was done for the primary outcome. Hypothesis testing of the other outcomes in the sequence was not done beyond the first non-significant result. For all subsequent and exploratory outcomes, reporting was restricted to the HR estimates and the nominal 95% CIs. Annualized incidence rates per 1000 patient-years of follow-up were calculated and the excess benefit or risk was estimated for outcomes with findings suggestive of benefit or harm. The analyses of albuminuria were based on individuals with progression or regression on at least one occasion with a sensitivity analysis performed for those with evidence of sustained progression or regression. Unless otherwise specified, on-treatment analysis (based upon patients who experienced a safety outcome while on study drug or within 30 days of study drug discontinuation) was the primary approach used for the safety assessments. The exception was for fracture, amputation, malignancy, and diabetic ketoacidosis outcomes where analyses included all events at any time point in all dosed patients. Effects of canagliflozin on continuous outcomes were assessed using mixed models that utilize all observed longitudinal data and assume missing at random. For all outcome analyses we tested the homogeneity of treatment effects across the two contributing trials.

### Results Summary

There were 10,142 trial participants (4330 CANVAS and 5812 CANVAS-R). 9734/10, 142 participants (96.0%) completed the study (i.e. were living participants assessed for safety and efficacy outcomes during the final follow-up window or had died prior to this). Vital status was confirmed for 10, 100/10, 142 participants (99.6%). Mean (median) follow up was 188.2 (126.1) weeks with comparable mean patient follow-up in randomized groups but greater mean follow-up in CANVAS (295.9 weeks) compared to CANVAS-R (108.0 weeks). There were 29.2% individuals assigned canagliflozin and 29.9% assigned placebo that discontinued randomized treatment prematurely.

Mean age of participants was 63.3 years, 35.8% were female, mean duration of diabetes was 13.5 years, mean eGFR was 76.5 ml/min/1.73 m² and mean UACR was 13.0 mg/mmol. There were 22.6% with microalbuminuria, 7.5% with macroalbuminuria and 65.6% with a history of atherosclerotic cardiovascular disease at baseline. 77% of CANVAS-R participants were up-titrated to the 300 mg dose by the last study visit. Patients were well-treated with other therapies for management of glycemia and cardiovascular risks. Baseline characteristics were balanced in the canagliflozin compared to placebo groups and directly comparable across CANVAS and CANVAS-R, as shown in Table 3 below.

TABLE 3
Baseline Characteristics in Active vs Placebo Groups

| | Canagliflozin | | Placebo | | Total | |
|---|---|---|---|---|---|---|
| | (n = 5795) | | (n = 4347) | | (N = 10142)* | |
| Age, years, mean (SD) | 63.2 | (8.3) | 63.4 | (8.2) | 63.3 | (8.3) |
| Female, n (%) | 2036 | (35.1) | 1597 | (36.7) | 3633 | (35.8) |
| Race, n (%) | | | | | | |
| White | 4508 | (77.8) | 3436 | (79.0) | 7944 | (78.3) |
| Asian | 777 | (13.4) | 507 | (11.7) | 1284 | (12.7) |
| Black or African American | 176 | (3.0) | 160 | (3.7) | 336 | (3.3) |
| Other^{†} | 334 | (5.8) | 244 | (5.6) | 578 | (5.7) |
| Current smoker, n (%) | 1020 | (17.6) | 786 | (18.1) | 1806 | (17.8) |
| History of hypertension, n (%) | 5188 | (89.5) | 3937 | (90.6) | 9125 | (90.0) |
| History of heart failure, n (%) | 803 | (13.9) | 658 | (15.1) | 1461 | (14.4) |
| Duration of diabetes, years, | 13.5 | (7.7) | 13.7 | (7.8) | 13.5 | (7.8) |
| mean (SD) | | | | | | |

Drug therapy, n (%)

| | | | | | | |
|---|---|---|---|---|---|---|
| Insulin | 2890 | (49.9) | 2205 | (50.7) | 5095 | (50.2) |
| Sulfonylurea | 2528 | (43.6) | 1833 | (42.2) | 4361 | (43.0) |
| Metformin | 4447 | (76.7) | 3378 | (77.7) | 7825 | (77.2) |
| GLP-1 receptor agonist | 222 | (3.8) | 185 | (4.3) | 407 | (4.0) |
| DPP-4 inhibitor | 697 | (12.0) | 564 | (13.0) | 1261 | (12.4) |
| Statin | 4329 | (74.7) | 3270 | (75.2) | 7599 | (74.9) |
| Antithrombotic | 4233 | (73.0) | 3233 | (74.4) | 7466 | (73.6) |
| RAAS inhibitor | 4645 | (80.2) | 3471 | (79.8) | 8116 | (80.0) |
| Beta blocker | 3039 | (52.4) | 2382 | (54.8) | 5421 | (53.5) |
| Diuretic | 2536 | (43.8) | 1954 | (45.0) | 4490 | (44.3) |

Microvascular disease history, n (%)

| | | | | | | |
|---|---|---|---|---|---|---|
| Retinopathy | 1203 | (20.8) | 926 | (21.3) | 2129 | (21.0) |
| Nephropathy | 994 | (17.2) | 780 | (17.9) | 1774 | (17.5) |
| Neuropathy | 1787 | (30.8) | 1323 | (30.4) | 3110 | (30.7) |

Atherosclerotic vascular disease history, n (%)^{‡}

| | | | | | | |
|---|---|---|---|---|---|---|
| Coronary | 3019 | (52.1) | 2261 | (52.0) | 5280 | (52.1) |
| Cerebrovascular | 1113 | (19.2) | 844 | (19.4) | 1957 | (19.3) |
| Peripheral | 1176 | (20.3) | 937 | (21.6) | 2113 | (20.8) |
| Any | 3976 | (68.6) | 3050 | (70.2) | 7026 | (69.3) |
| CV disease history, n (%)^{§} | 3756 | (64.8) | 2900 | (66.7) | 6656 | (65.6) |
| History of amputation, n (%) | 136 | (2.3) | 102 | (2.3) | 238 | (2.3) |
| Body mass index, kg/m², | 31.9 | (5.9) | 32.0 | (6.0) | 32.0 | (5.9) |
| mean (SD) | | | | | | |
| Systolic BP, mmHg, | 136.4 | (15.8) | 136.9 | (15.8) | 136.6 | (15.8) |
| mean (SD) | | | | | | |
| Diastolic BP, mmHg, | 77.6 | (9.6) | 77.8 | (9.7) | 77.7 | (9.7) |
| mean (SD) | | | | | | |
| Glycated hemoglobin, | 8.2 | (0.9) | 8.2 | (0.9) | 8.2 | (0.9) |
| %, mean (SD) | | | | | | |
| Total cholesterol, mmol/l, | 4.4 | (1.1) | 4.4 | (1.2) | 4.4 | (1.2) |
| mean (SD) | | | | | | |
| Triglycerides, mmol/l, | 2.0 | (1.3) | 2.0 | (1.5) | 2.0 | (1.4) |
| mean (SD) | | | | | | |
| HDL cholesterol, mmol/l, | 1.2 | (0.3) | 1.2 | (0.3) | 1.2 | (0.3) |
| mean (SD) | | | | | | |
| LDL cholesterol, mmol/l, | 2.3 | (0.9) | 2.3 | (0.9) | 2.3 | (0.9) |
| mean (SD) | | | | | | |
| LDL/HDL cholesterol | 2.0 | (0.9) | 2.0 | (0.9) | 2.0 | (0.9) |
| ratio, mean (SD) | | | | | | |
| eGFR, ml/min/1.73 m², | 76.7 | (20.3) | 76.2 | (20.8) | 76.5 | (20.5) |
| mean (SD)^{∥} | | | | | | |
| Albumin:creatinine ratio, | 12.3 | (49.0) | 14.0 | (51.0) | 13.0 | (49.9) |
| mg/mmol, mean (SD) | | | | | | |
| Normoalbuminuria, n (%) | 4012 | (69.9) | 2995 | (69.8) | 7007 | (69.8) |
| Microalbuminuria, n (%) | 1322 | (23.0) | 944 | (22.0) | 2266 | (22.6) |
| Macroalbuminuria, n (%)** | 406 | (7.1) | 354 | (8.3) | 760 | (7.6) |

SD, standard deviation;
GLP-1, glucagon-like peptide-1;
DPP-4, dipeptidyl peptidase-4,
RAAS, renin angiotensin aldosterone system;
BP, blood pressure;
HDL, high-density lipoprotein;
LDL, low-density lipoprotein,
eGFR, estimated glomerular filtration rate.
*One participant was randomized at 2 different sites and only the first randomization is included in the ITT analysis set.
^{†}Includes American Indian or Alaska Native, Native Hawaiian or other Pacific Islander, multiple, other, and unknown.
^{‡}Some participants had ≥1 type of atherosclerotic disease.
^{§}As defined in the protocol.
^{∥}Values for eGFR categories calculated based on N of 5794 for canagliflozin, 4346 for placebo, and 10140 for the total population.
^{¶}Values for albuminuria categories calculated based on N of 5740 for canagliflozin, 4293 for placebo, and 10033 for the total population.

### Intermediate Markers of Cardiovascular Risk

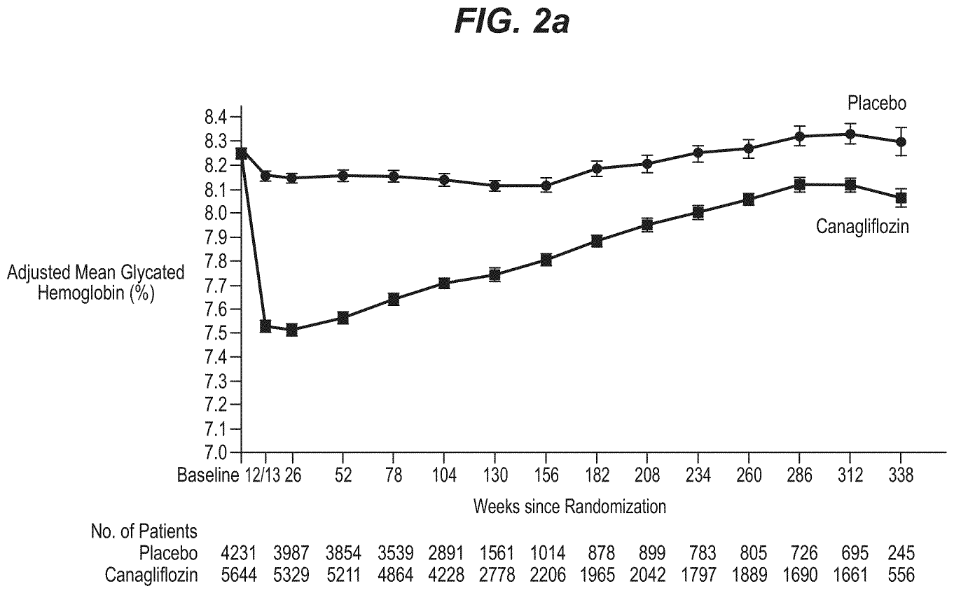

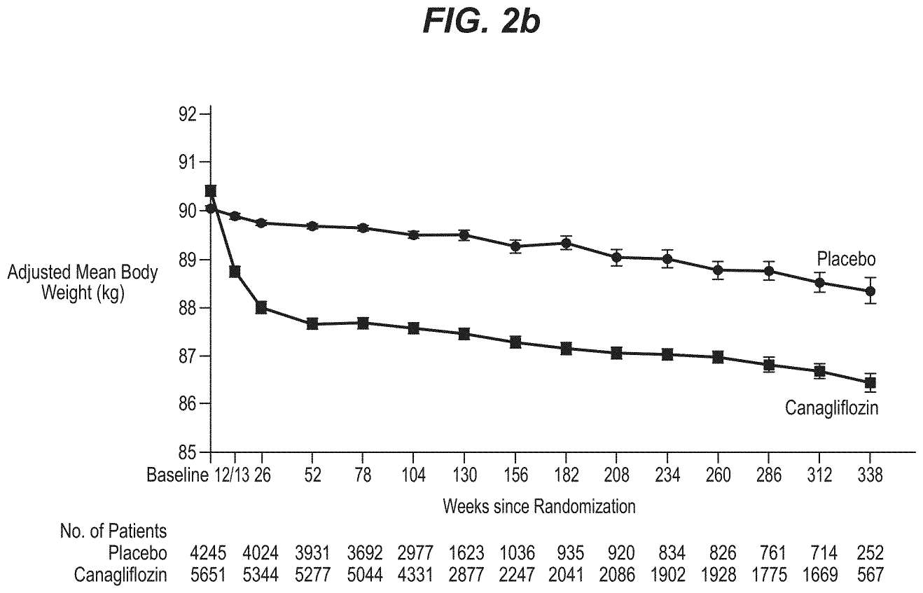

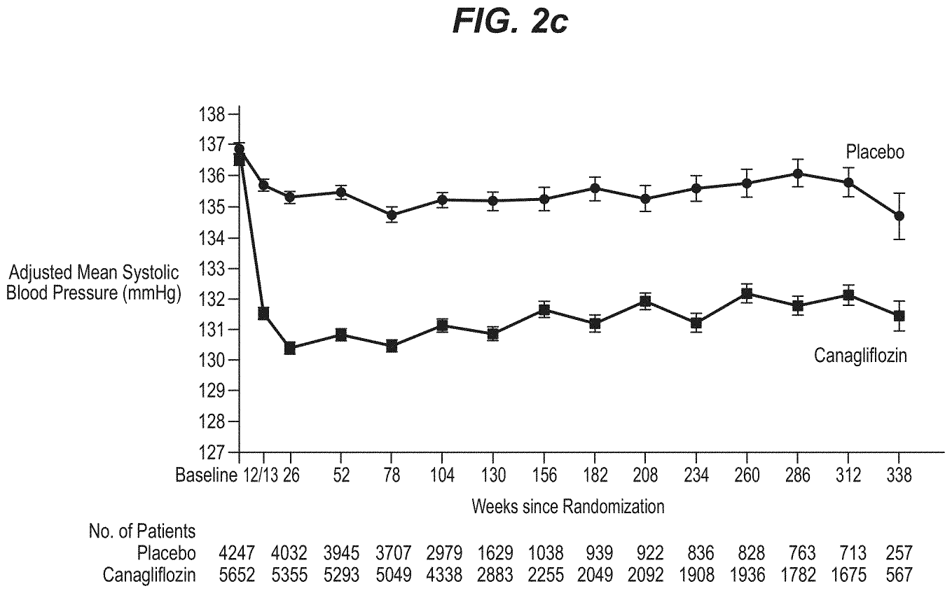

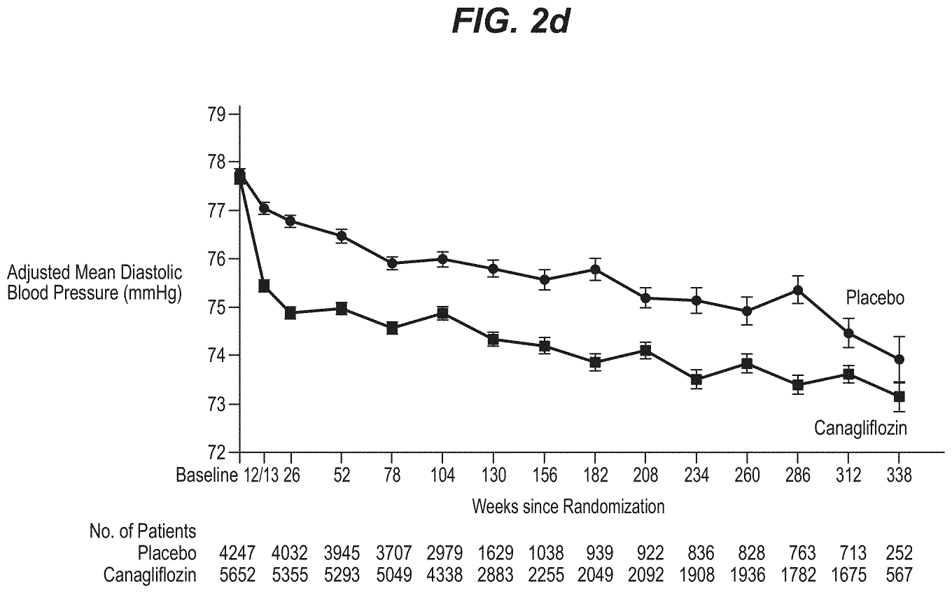

The effect of canagliflozin on glycated hemoglobin, body weight, systolic and diastolic blood pressure in the combined CANVAS and CANVAS-R clinical trials (n=10,142) was as shown in FIGS. 2a) through 2d).

For canagliflozin compared to placebo, the mean difference in glycated hemoglobin was −0.58% (95% CI −0.61 to −0.56%), the mean difference in body weight was −1.60 kg (95% CI −1.70 to −1.51 kg) and the mean difference in systolic blood pressure was −3.93 mmHg (95% CI −4.30 to −3.56 mmHg), all P<0.001. Use of other antihyperglycemic agents during follow-up was 9.3% lower (95% CI −11.0% to −7.6%) in the canagliflozin compared to placebo group.

### Cardiovascular Outcomes, Death and Hospitalizations

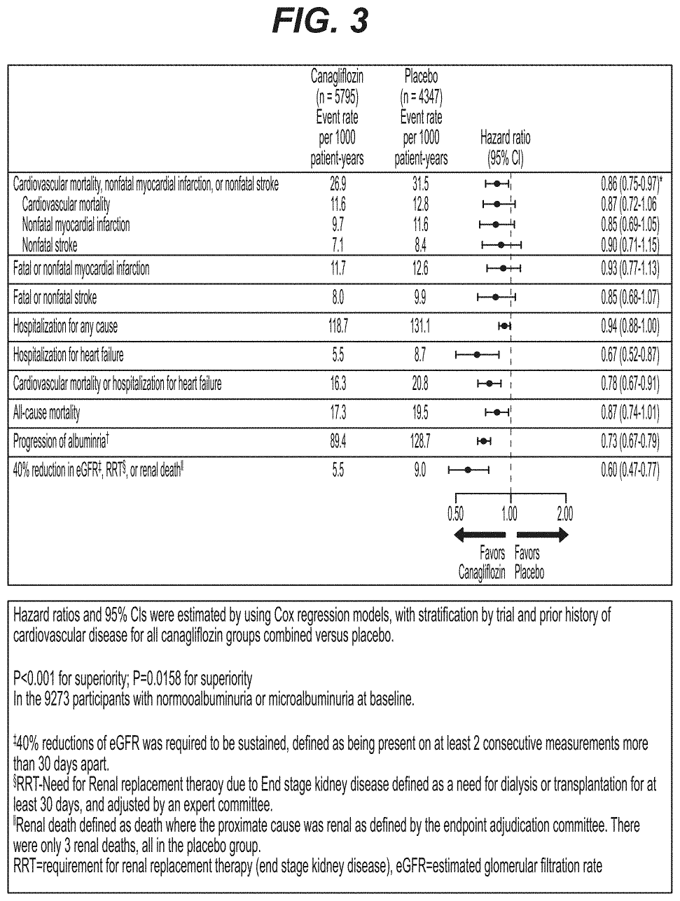

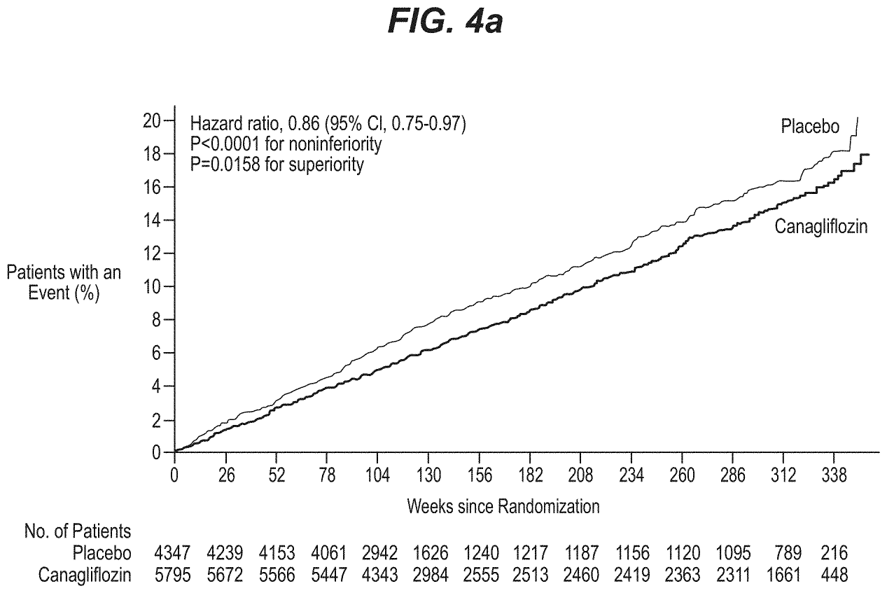

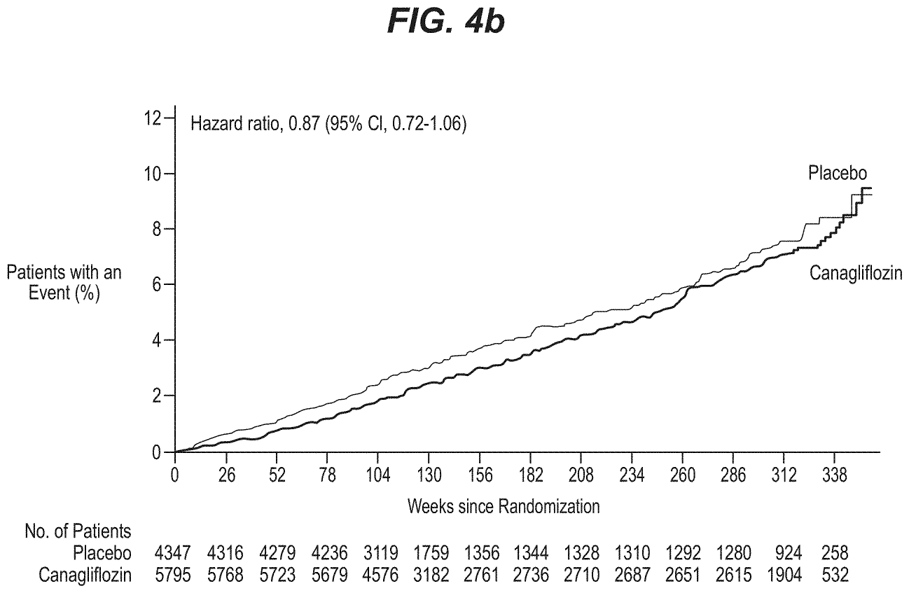

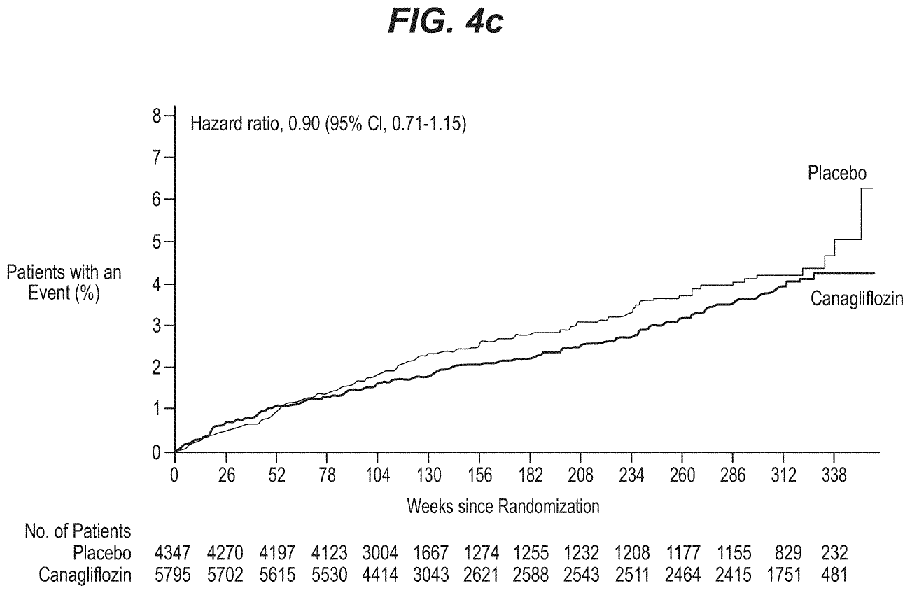

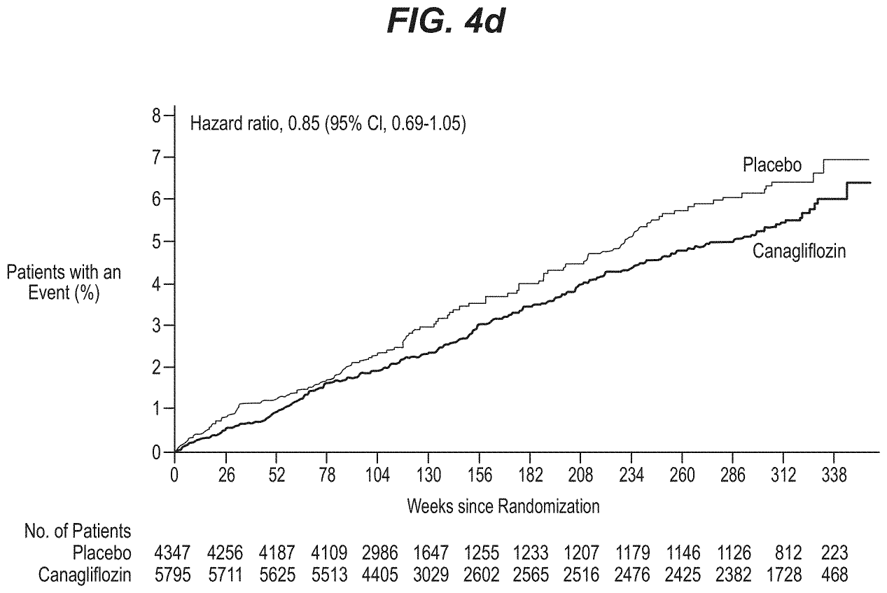

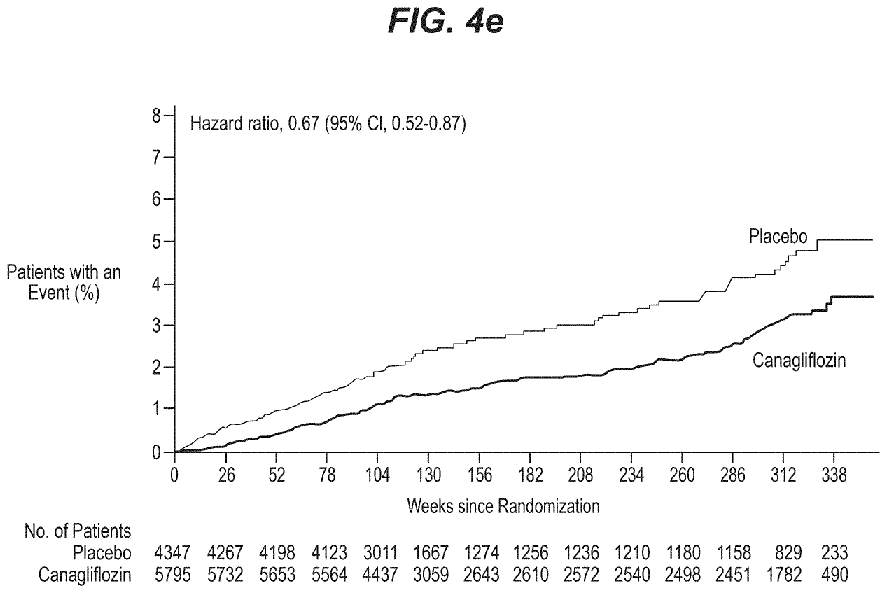

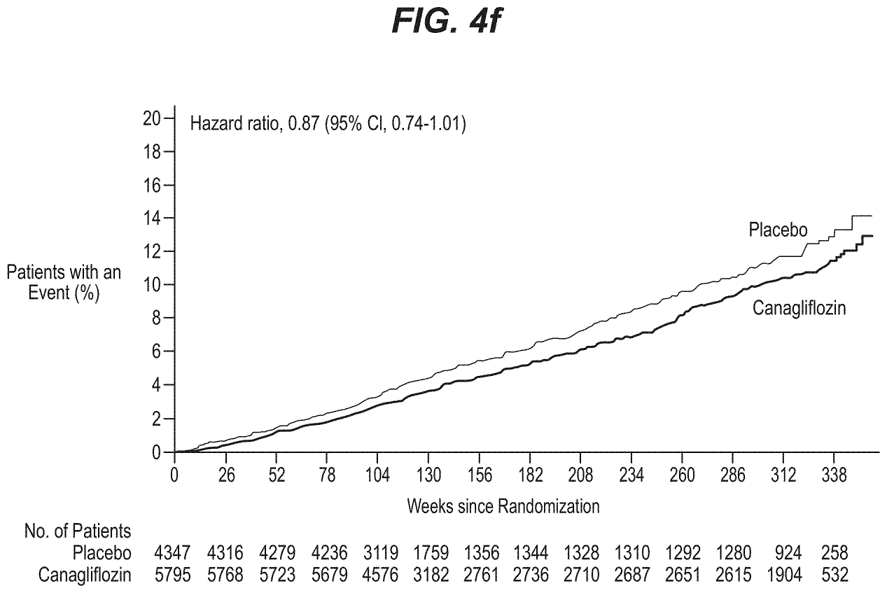

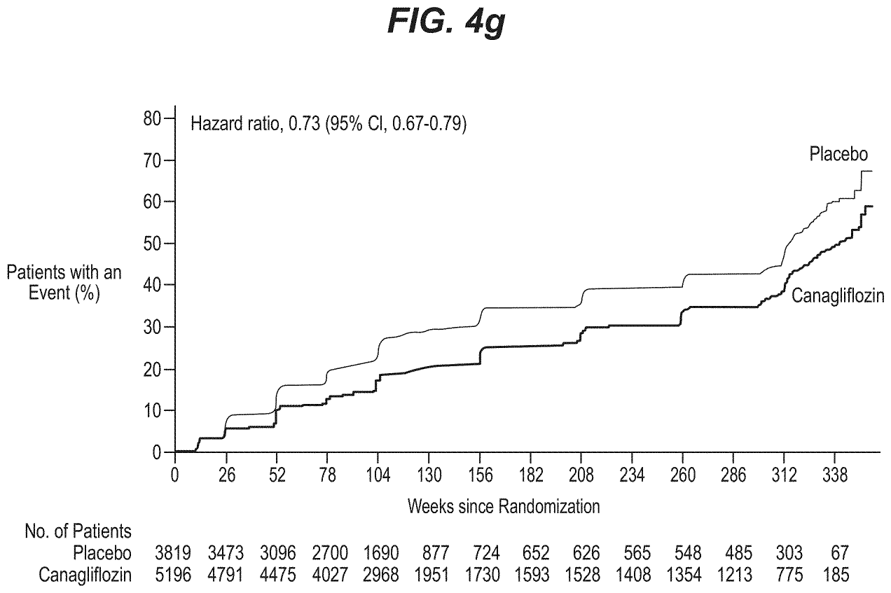

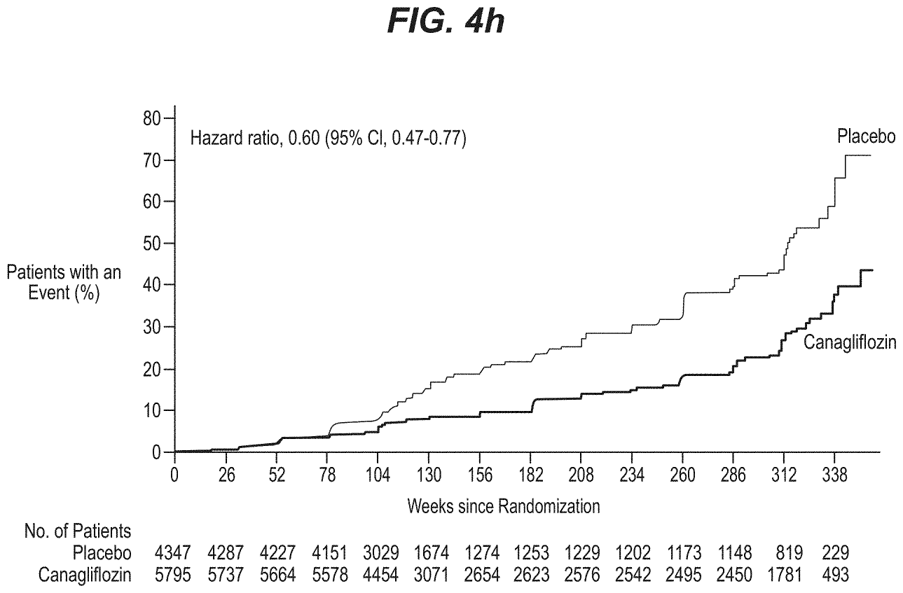

Significantly fewer primary outcome events (the composite of cardiovascular mortality, nonfatal myocardial infarction, or nonfatal stroke) occurred in the canagliflozin group than the placebo group (26.9 vs. 31.5/1000 patient-years, HR (hazard ratio) 0.86, 95% CI 0.75 to 0.97; P<0.0001 for noninferiority; P=0.0158 for superiority). The effects on the primary outcome were the same when imputation for missing events was performed (HR 0.85, 95% CI 0.75 to 0.97). There were broadly consistent effects across a broad range of prespecified subgroups except for subsets defined by baseline use of diuretic or not (P=0.0001 for homogeneity). Superiority was not demonstrated for the first secondary outcome in the testing sequence (all-cause mortality, P=0.245) and hypothesis testing was discontinued. Nominal effect estimates for the fatal secondary outcomes are HR 0.87 (95% CI 0.74 to 1.01) for all-cause mortality and HR 0.87 (95% CI 0.72 to 1.06) for cardiovascular death. There was no evidence of differences in effects between the CANVAS and CANVAS-R trials for the primary, fatal, or exploratory cardiovascular outcomes. Effect of canagliflozin versus placebo on cardiovascular outcomes were as shown in FIG. 3, FIGS. 4a) through 4h) (as a function of time) and in Table 4, below.

TABLE 4
Effect on Cardiovascular Outcomes

| | | | |
|---|---|---|---|
| | | Hazard ratio | |
| Canagliflozin | Placebo | (95% confidence | P |
| n/N | n/N | interval) | value^{‡} |

Cardiovascular mortality, nonfatal
myocardial infarction, or nonfatal stroke

| | | | | |
|---|---|---|---|---|
| CANVAS | 425/2888 | 233/1442 | 0.88 (0.75-1.03) | |
| CANVAS-R | 160/2907 | 193/2905 | 0.82 (0.66-1.01) | |
| CANVAS Program | 585/5795 | 426/4347 | 0.86 (0.75-0.97) | 0.5980 |

Total mortality

| | | | | |
|---|---|---|---|---|
| CANVAS | 301/2888 | 175/1442 | 0.84 (0.70-1.01) | |
| CANVAS-R | 99/2907 | 106/2905 | 0.92 (0.70-1.21) | |
| CANVAS Program | 400/5795 | 281/4347 | 0.87 (0.74-1.01) | 0.5675 |

Cardiovascular mortality

| | | | | |
|---|---|---|---|---|
| CANVAS | 207/2888 | 115/1442 | 0.88 (0.70-1.10) | |
| CANVAS-R | 61/2907 | 70/2905 | 0.86 (0.61-1.22) | |
| CANVAS Program | 268/5795 | 185/4347 | 0.87 (0.72-1.06) | 0.9387 |

Cardiovascular mortality or hospitalization for heart failure

| | | | | |
|---|---|---|---|---|
| CANVAS | 269/2888 | 158/1442 | 0.82 (0.67-0.99) | |
| CANVAS-R | 95/2907 | 130/2905 | 0.72 (0.55-0.94) | |
| CANVAS Program | 364/5795 | 288/4347 | 0.78 (0.67-0.91) | 0.4584 |

Nonfatal myocardial infarction

| | | | | |
|---|---|---|---|---|
| CANVAS | 152/2888 | 86/1442 | 0.85 (0.65-1.11) | |
| CANVAS-R | 63/2907 | 73/2905 | 0.85 (0.61-1.19) | |
| CANVAS Program | 215/5795 | 159/4347 | 0.85 (0.69-1.05) | 0.9777 |

Nonfatal stroke

| | | | | |
|---|---|---|---|---|
| CANVAS | 106/2888 | 53/1442 | 0.97 (0.70-1.35) | |
| CANVAS-R | 52/2907 | 63/2905 | 0.82 (0.57-1.18) | |
| CANVAS Program | 158/5795 | 116/4347 | 0.90 (0.71-1.15) | 0.4978 |

Hospitalization for heart failure

| | | | | |
|---|---|---|---|---|
| CANVAS | 85/2888 | 53/1442 | 0.77 (0.55-1.08) | |
| CANVAS-R | 38/2907 | 67/2905 | 0.56 (0.38-0.83) | |
| CANVAS Program | 123/5795 | 120/4347 | 0.67 (0.52-0.87) | 0.2359 |

^{‡}P value for homogeneity CANVAS and CANVAS-R.

### Renal Outcomes

Progression of albuminuria grade occurred less frequently in participants randomized to canagliflozin than to placebo (89.4 vs. 128.7/1000 patient-years) corresponding to a HR of 0.73 (95% CI 0.67 to 0.79) with greater effects in CANVAS-R (HR 0.64, 95% CI 0.57 to 0.73) compared to CANVAS (HR 0.80, 95% CI 0.72 to 0.90) (p homogeneity=0.02). The composite outcome of sustained 40% reduction in eGFR, end-stage kidney disease or renal death occurred less frequently among participants randomized to canagliflozin compared to placebo (5.5 vs. 9.0/1000 patient-years) corresponding to a HR of 0.60 (95% CI 0.47 to 0.77). Effect of canagliflozin versus placebo on renal outcomes were as shown in Table 5 below.

TABLE 5
Effect on Renal Outcomes

| | | | |
|---|---|---|---|
| | | Hazard ratio | |
| Canagli- | | (95% | |
| flozin | Placebo | confidence | P |
| n/N | n/N | interval) | value^{‡} |

Progression of albuminuria

| | | | | |
|---|---|---|---|---|
| CANVAS | 895/2655 | 479/1301 | 0.80 (0.72-0.90) | |
| CAN VAS-R | 446/2541 | 635/2518 | 0.64 (0.57-0.73) | |
| CANVAS | 1341/5196 | 1114/3819 | 0.73 (0.67-0.79) | 0.0184 |
| Program | | | | 0.8750^{§} |

Regression of albuminuria

| | | | | |
|---|---|---|---|---|
| CANVAS | 434/786 | 162/400 | 1.56 (1.30-1.87) | |
| CANVAS-R | 451/893 | 283/857 | 1.80 (1.55-2.09) | |
| CANVAS | 885/1679 | 445/1257 | 1.70 (1.51-1.91) | 0.4587 |
| Program | | | | |

40% reduction in eGFR,* RRT, or renal death**

| | | | | |
|---|---|---|---|---|
| CANVAS | 91/2888 | 78/1442 | 0.56 (0.41-0.75) | |
| CANVAS-R | 33/2907 | 47/2905 | 0.71 (0.45-1.11) | |
| CANVAS | 124/5795 | 125/4347 | 0.60 (0.47-0.77) | 0.3868 |
| Program | | | | |

40% reduction in eGFR,* RRT, renal death** or macroalbuminuria

| | | | | |
|---|---|---|---|---|
| CANVAS | 292/2888 | 214/1442 | 0.64 (0.54-0.76) | |
| CANVAS-R | 112/2907 | 229/2905 | 0.48 (0.38-0.60) | |
| CANVAS | 404/5795 | 443/4347 | 0.57 (0.50-0.66) | 0.0500 |
| Program | | | | |

40% reduction in eGFR,* RRT, renal or CV death**

| | | | | |
|---|---|---|---|---|
| CANVAS | 286/2888 | 186/1442 | 0.74 (0.62-0.89) | |
| CANVAS-R | 93/2907 | 114/2905 | 0.82 (0.62-1.08) | |
| CANVAS | 379/5795 | 300/4347 | 0.77 (0.66-0.89) | 0.5503 |
| Program | | | | |

*40% reductions of eGFR and doubling of creatinine were required to be sustained, defined as being present on at least 2 consecutive measurements more than 30 days apart
**RRT—Need for Renal replacement therapy due to End stage kidney disease defined as a need for dialysis or transplantation for at least 30 days, and adjudicated by an expert committee. Renal death defined as death where the proximate cause was renal as defined by the endpoint adjudication committee. There were only 3 renal deaths, all in the placebo group. Results where 40% reduction in eGFR were substituted by doubling serum creatinine were not substantively different.
^{‡}P value for homogeneity CANVAS and CANVAS-R.
^{§}Gail-Simon P value.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method for reducing or preventing one or more cardiovascular events, each cardiovascular event independently selected from the group consisting of cardiovascular hospitalization, non-fatal myocardial infarction, non-fatal ischemia or stroke, and cardiovascular mortality, comprising administering to a patient in need thereof, a therapeutically effective amount of canagliflozin;
wherein the patient in need thereof is a patient diagnosed with Type II diabetes mellitus; and wherein the patient further exhibits symptoms of or is diagnosed with one or more concomitant or comorbid cardiovascular risk factors or cardiovascular disease.

2. A method for reducing or preventing one or more major adverse cardiovascular events (MACE), each major adverse cardiac event independently selected from the group consisting of cardiovascular death, non-fatal myocardial infarction, cardiac arrhythmia, and non-fatal stroke, comprising administering to a patient in need thereof a therapeutically effective amount of canagliflozin;
wherein the patient in need thereof is a patient diagnosed with Type II diabetes mellitus; and wherein the patient further exhibits symptoms of or is diagnosed with one or more concomitant or comorbid cardiovascular risk factors or cardiovascular disease.

3. A method as in claim 1, wherein the patient diagnosed with Type II diabetes mellitus has a measured HbA1c in the range of ≥7.0% and ≤10.5%.

4. A method as in claim 1, wherein the patient is further diagnosed with microalbuminuria or macroalbuminuria.

5. A method as in claim 1, wherein the one or more cardiovascular risk factor is selected from the group consisting of obesity, hypertension, hyperlipidemia, elevated triglycerides, microalbuminuria and macroalbuminuria.

6. A method as in claim 1, wherein the one or more cardiovascular risk factor is selected from the group consisting of obesity, hypertension, hyperlipidemia and elevated triglycerides.

7. A method as in claim 1, wherein the cardiovascular disease is selected from the group consisting of history of non-fatal myocardial infarction, history of non-fatal stroke, peripheral artery disease, hypertensive heart disease, ischemic heart disease, coronary vascular disease, peripheral vascular disease, cerebrovascular disease, cardiac arrhythmia (other than sinus tachychardia), cardiomyopathy, angina, heart failure and coronary valve disease.

8. A method as in claim 1, wherein the cardiovascular risk or cardiovascular disease is one or more selected from the group consisting of heart failure, coronary vascular disease, cerebrovascular disease, peripheral vascular disease and hypertension.

9. (canceled)

10. A method as in claim 1, wherein the canagliflozin is administered in an amount in the range of from about 50 to about 500 mg.

11. A method as in claim 1, wherein the canagliflozin is administered in an amount in the range of from about 100 to about 300 mg.

12. A method as in claim 1, which is safe and effective.

13. A method as in claim 1, wherein the method reduces a patient's hazard ratio (HR) for a cardiovascular event selected from the group consisting of cardiovascular hospitalization, non-fatal myocardial infarction, non-fatal stroke and cardiovascular death, to a value in the range of from about 0.95 to about 0.60.

14. A method as in claim 1, wherein the method reduces the predicted severity of an adverse cardiovascular event or decrease the predicted mortality from an adverse cardiovascular event, or decrease the progression of cardiovascular disease.

15. A method as in claim 1, wherein the method increases the predicted life expectancy of the subject, the predicted time period between adverse cardiovascular events, or the effectiveness of a cardiovascular intervention in the subject.

16. A method as in claim 1, wherein the method increases the time until first incidence of one or more events selected from the group consisting of cardiovascular death, non-fatal myocardial infarction, stroke, non-fatal stroke, nonfatal hospitalization and cardiovascular mortality.

17. A method as in claim 1, wherein the patient has one or more of the following characteristics at the time of treatment: a) existing cardiovascular disease or a high likelihood of cardiovascular disease; b) congestive heart failure; c) family history of cardiovascular disease; d) current smoker; e) genetically predisposed to cardiovascular diseases; f) has or has had cardiac arrhythmia; g) has or has had atrial fibrillation, ventricular fibrillation, or tachyarrhythmia; h) does not have sinus tachycardia; i) has unstable angina; j) has hypertension; k) has had a stroke or is at increased risk of stroke; l) has an aneurysm; and/or m) has elevated triglycerides, elevated LDL, and/or low HDL.

18. A method as in claim 1, wherein the patient has either a confirmed diagnosis of cardiovascular disease or a high likelihood of cardiovascular disease, and further, said patient has at least one of: a) a history of documented myocardial infarction; b) a history of coronary revascularization; c) a history of carotid or peripheral revascularization; d) angina with ischemic changes; e) ECG changes on a graded exercise test; f) positive cardiac imaging study; g) ankle brachial index<0.9; and/or h) >50% stenosis of a coronary artery, carotid artery, or lower extremity artery.

19. A method as in claim 1, wherein the patient has had one or more of the following: (a) a myocardial infarction; (b) a history of angina pectoris; (c) a history of cerebrovascular disease; (d) a history of stroke; (e) a history of tachycardia other than sinus tachycardia; or (f) a planned bariatric surgery, cardiac surgery, or coronary angioplasty.
